(19) United States
(12) Patent Application Publication
(10) Pub. No.: US 2022/0392609 A1
(43) Pub. Date: Dec. 8, 2022

(54) PERSONALIZED FOOD RECOMMENDATIONS BASED ON SENSED BIOMARKER DATA

(71) Applicant: Medtronic MiniMed, Inc., Northridge, CA (US)

(72) Inventors: Yaron Hadad, San Francisco, CA (US); Jonathan Lipnik, Ramat Gan (IL); Orr Mandelbaum, San Francisco, CA (US)

(21) Appl. No.: 17/339,467

(22) Filed: Jun. 4, 2021

(51) Int. Cl. G16H 20/60; A61B 5/00; G16H 50/20; G06N 20/00; G09B 19/00

(52) U.S. Cl. CPC G16H 20/60; A61B 5/4866; A61B 5/7267; A61B 5/4836; G16H 50/20; G06N 20/00; G09B 19/0092

(57) ABSTRACT

Device, systems, and techniques for supporting a patient's diabetes management with food item recommendations are described in this disclosure. The device, systems, and techniques may be configured to execute a training process for a model to predict a patient nutrition state of a patient based on a predetermined food item consumed by the patient within a time period. The training process is further configured to determine an estimated biomarker level based on the predetermined food item profile having a set of nutritional attributes for the food item and the model; receive an actual biomarker level of the patient after the patient consumes the food item within the time period; and calibrate the model based on comparing the estimated biomarker level to the actual biomarker level; repeat the training process for one or more food items of a set of predetermined food items; and output the trained model.

### TECHNICAL FIELD

The disclosure relates to medical systems and, more particularly, to medical systems for therapy for diabetes.

### BACKGROUND

A patient with diabetes typically receives insulin from an insulin delivery device (e.g., a pump or injection device) to control the glucose level in his or her bloodstream. Naturally produced insulin may not adequately control the glucose level in the bloodstream of a diabetes patient due to insufficient production of insulin and/or due to insulin resistance. To control the glucose level, a patient's therapy routine may include basal dosages and bolus dosages of insulin. Basal dosages tend to keep glucose levels at consistent levels during periods of fasting. Bolus dosages may be delivered to the patient specifically at or near mealtimes or other times where there may be a relatively fast change in glucose level.

### SUMMARY

Devices, systems, and techniques for managing biomarker levels in a patient are described. Some example devices, systems, and techniques provide the patient with personalized biomarker (e.g., glucose) level management. This may involve a kit of pre-determined (e.g., baseline) foods to prepare the example devices, systems, and techniques to predict the patient's specific physiological response to other food items. The patient consumes one or more pre-determined foods in similar contexts (e.g., after waking up or after fasting) and the example devices, systems, and techniques calibrate a therapy delivery device (e.g., a glucose delivery device) to provide an appropriate amount and/or type of therapy (e.g., insulin) and/or at a correct therapy schedule.

The pre-determined foods may have precise standardized nutrition profiles, enabling a direct comparison between users of a same or similar biomarker sensor (e.g., continuous glucose monitor (CGM)) who are consuming the same food item in similar situations. Having a pre-defined nutritional breakdown of a food item allows for decoupling of how different ingredients affect people in general (e.g., biologically), and then, mapping that food item to each user's individual physiological response to consuming that food item. By mapping the pre-determined foods in the kit to in each user's biomarker (e.g., glucose) level changes, some example devices, systems, and techniques may use those mappings to extrapolate each user's individual physiological response to other food items. Some example devices, systems, and techniques leverage the above mappings into more granular mappings between a specific ingredient and a personalized biomarker level prediction (i.e., a “FoodPrint”).

Some example devices, systems, and techniques rely on such mappings to train a machine learning model to accurately predict personalized biomarker (e.g., glucose) levels for any given food item with a known and/or pre-determined nutritional composition. Even if the nutritional composition is unknown or ambiguous, some examples invoke various mechanisms to determine at least some nutrition information (e.g., one or more nutritional facts such as a calorie count). Once trained, the trained machine learning model may benefit the patient with personalized food item recommendation based on that food item's impact on the patient's physiology. Given at least some of a candidate food item's nutritional composition, some examples leverage the trained machine learning model to evaluate the candidate food item as a potential recommendation. Some examples are provided with an inventory of the patient's readily available food items and after applying the machine learning model to nutrition information for these food items, at least one food item may be recommended for the patient's consumption in the near future.

In one example, the disclosure describes a system comprising: memory configured to store a model; and processing circuitry communicatively coupled to the memory, wherein the processing circuitry is configured to: execute a training process for the model to predict a patient nutrition state of a patient based on a predetermined food item consumed by the patient within a time period, wherein to execute the training process, the processing circuitry is further configured to: determine an estimated biomarker level based on the predetermined food item profile having a set of nutritional attributes for the food item and the model; receive an actual biomarker level of the patient after the patient consumes the food item within the time period; and calibrate the model based on comparing the estimated biomarker level to the actual biomarker level; repeat the training process for one or more food items of a set of predetermined food items; and output the trained model.

In one example, the disclosure describes a system comprising: memory configured to store a trained model; and processing circuitry communicatively coupled to the delivery device, wherein the processing circuitry is configured to: apply the trained model to one or more food item profiles to generate a predicted patient nutrition state for each of the one or more food item profiles, wherein the trained model generates each predicted patient nutrition state of a patient based on a set of nutritional attributes for a corresponding food item; select, without patient input, one or more food items associated with the one or more food item profiles to recommend for consumption by the patient based at least on predicted patient nutrition states of the patient corresponding to the one or more food item profiles, wherein the predicted patient nutrition state comprises at least one estimated biomarker level expected to be within at least one desired range if the patient consumes the selected food item; and generate, for display, output data indicating the selected one or more food items.

In one example, the disclosure describes A method performed by a medical system having memory for storing a trained model, the method comprising: applying, by processing circuitry of the medical system, a trained model to one or more food item profiles to generate a predicted patient nutrition state of a patient for each of the one or more food item profiles; selecting, by the processing circuitry, one or more food items to recommend for consumption by the patient based at least on the predicted patient nutrition state after the patient consumes each of the one or more selected food items, wherein the predicted patient nutrition state comprises at least one estimated biomarker level expected to be within at least one desired range if the patient consumes the one or more selected food items; and generating, by the processing circuitry, output data for display indicating the selected one or more food items.

The details of one or more aspects of the disclosure are set forth in the accompanying drawings and the description below. Other features, objects, and advantages of this disclosure will be apparent from the description and drawings, and from the claims.

### BRIEF DESCRIPTION OF DRAWINGS

FIG. 1 is a block diagram illustrating an example system for delivering or guiding therapy dosage, in accordance with one or more examples described in this disclosure.

FIG. 2 is a block diagram illustrating another example system for delivering or guiding therapy dosage, in accordance with one or more examples described in this disclosure.

FIG. 3 is a block diagram illustrating another example system for delivering or guiding therapy dosage, in accordance with one or more examples described in this disclosure.

FIG. 4 is a block diagram illustrating an example of a patient device, in accordance with one or more examples described in this disclosure.

FIG. 5 is a block diagram illustrating an example of a wearable device, in accordance with one or more examples described in this disclosure.

FIG. 6A and FIG. 6B are illustrations of a packaging structure for a kit, in accordance with one or more examples described in this disclosure.

FIGS. 7A-C are each flow diagrams that, in combination, depict an example method, in accordance with one or more examples described in this disclosure.

### DETAILED DESCRIPTION

Devices, systems, and techniques for supporting a patient's diabetes management with food item recommendations are described in this disclosure. The present disclosure describes food item recommendations that are personalized for the patient. Some example devices, systems, and techniques leverage machine learning technologies to identify an appropriate food item to recommend to the patient. A food item may be appropriate to recommend the patient, for example, if that food item is determined to be healthy and/or safe for the patient to consume. In another example, an appropriate food item to recommend the patient may also benefit the patient's current therapy, for example, by not interfering with any scheduled treatment. As described herein, the patient's current therapy may account for the patient's typical diet. Therefore, a food item is inappropriate and therefore, is not recommended if the food item deviates from the patient's diet and may cause the next scheduled treatment (e.g., insulin dose) to be ineffective or less effective.

Because the patient derives food energy from carbohydrates, fats, and proteins as well as from organic acids, polyols, and ethanol present in food items that the patient eats, the nutritional composition of each food item impacts that patient's biomarkers. Examples of the biomarkers include glucose level, ketone levels, lactic acid levels, and the like. However, the impact of a particular food on a biomarker may vary between individuals and also vary for the same individual. For example, the change in glucose level due to eating an apple may vary between individuals, especially those with diabetes. Also, in some cases, the change in glucose level due to eating an apple may vary in the same individual based on various factors such as time of day, stress level, physical activity levels, lack of sleep, etc.

A personalized machine learning model as described herein is applied to food item information to determine if consuming that food item is likely to cause an increase or a decrease in a biomarker level of interest. For ease, the disclosure describes example techniques in the context of managing glucose level. However, the techniques are not so limited, and may be applicable to managing various biomarkers (e.g., keeping biomarkers within a range) such as to optimize health and nutrition. As an example, the example techniques may be applicable to generating personalized food recommendations for an individual trying to keep ketone levels within a certain range. As another example, the example techniques may be applicable to an athlete trying to keep lactic acid within a certain range.

The present disclosure also describes example techniques to sufficiently train a personalized model for the patient's benefit. Once fully trained, the personalized model may accurately estimate changes (e.g., increases or decreases) to the patient's biomarker levels. In some examples, by modeling the patient's specific physiological response to a food item's nutritional composition, the personalized model may predict certain biomarker levels for determining whether the biomarker levels are expected to be within a healthy range.

To illustrate the above technique by way of example, the training technique may apply the above personalized model to input features corresponding to an example food item's nutritional composition and estimate a biomarker level of interest based on that nutrition information. One or more sensors provide measurements including the patient's actual biomarker level at a point in time after the patient consumes the example food item. The training technique may compare the estimated and actual biomarker levels and based on that comparison, calibrate the personalize model to better estimate the patient's biomarker levels, for example, by adjusting model data (e.g., model parameters or hyperparameters). The training technique may further calibrate the personalized model by repeating the above comparison for a number of food items before outputting a trained personalized machine learning model.

In one or more examples, because the impact of food on biomarkers may be different for each individual, an individual may be instructed to consume a specific food item within a preselected set of foods for determining the impact of food on biomarkers for that individual. For instance, while training the personalized machine learning model, the patient may be provided with several food kits (e.g., seven kits), with each kit including an edible consumable (e.g., a bar or a beverage) with a particular nutritional composition. On each day, such as in the morning after a fasting period due to sleep, the individual may consume one of the consumables. A glucose sensor connected to the patient may measure the change in glucose level due to the consumption of each consumable. Because each consumable has a unique nutritional composition, it may be possible to determine generally the impact of food on biomarkers for the individual over many different types of foods. In some examples, training of the personalized machine learning model may include the patient consuming two or more food kits (e.g., the same or different food kits) within a 24-hour period. For example, training of the personalized machine learning model may include the patient consuming a combination of a “breakfast” kit, one or more “snack” kits, a “lunch” kit, and/or a dinner kit. In this way, the personalized machine learning model may be trained to determine how combination of food items may affect the patient's biomarker levels over time.

That is, the edible consumables in the kits may be considered as baseline foods from which a computing device may determine the impact on the patient's biomarkers after the patient consumes different foods. Based on the impact of food on biomarkers, and the goals of an individual (e.g., weight loss, increased muscle, keeping glucose level within a desired range, etc.), it may be possible to determine personalized food recommendations to achieve those goals.

For example, a computing device may utilize the nutritional composition of the pre-determined baseline foods and the resulting biomarker information to train a machine learning model and generate a trained model for deployment to patient devices. The input for the trained model may be various food items or food types that an individual might want to consume, and the output may be a prediction of the values of one or more biomarkers (e.g., including overtime) if the individual were to consume those foods. Based on the prediction of biomarkers if the individual were to consume particular foods and the goals of the individual, the computing device may generate personalized meal recommendations.

The computing device may store a volume measurement for each baseline food item and then, use that volume measurement to estimate the baseline food item's nutritional content and/or portion size (e.g., serving size). To determine how much the patient's glucose level will likely change by consuming the particular food item given the estimated nutritional content and/or portion size, patient device and/or cloud may employ a mathematical function or probability model that is configured to accept, as input variables, factors corresponding to the estimated nutritional content and/or portion size and generate, as output data, an expected amount of increase or decrease in the glucose level of the patient. In some examples, the mathematical function or probability model may be static and pre-determined by health professionals. In other examples, the patient device and/or the cloud service may employ machine learning techniques to dynamically adjust the mathematical function or probability model in response to feedback (e.g., “learning”) in a manner that improves upon an accuracy of the mathematical function or probability model.

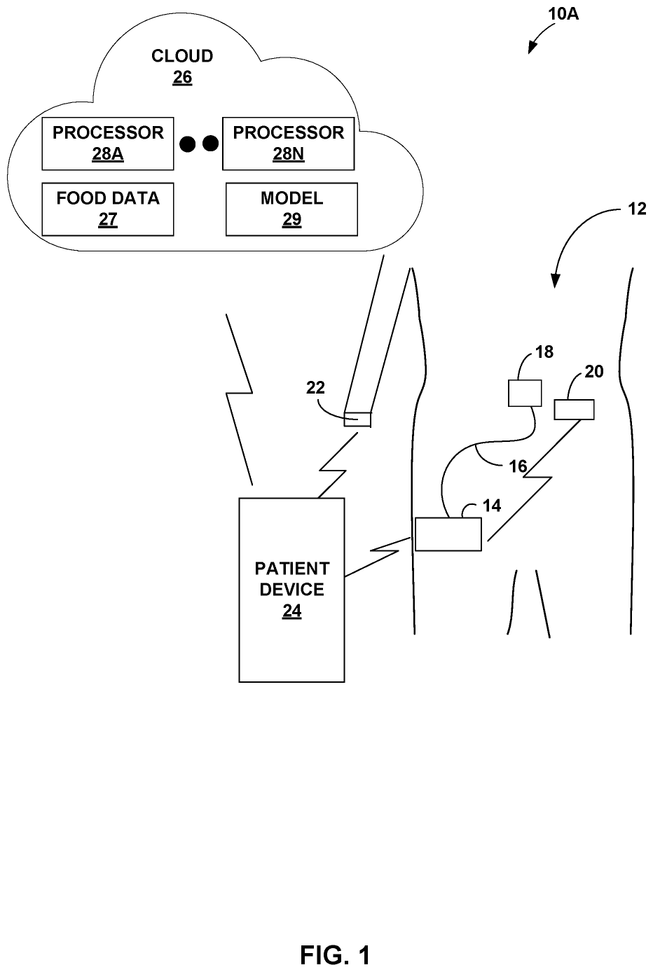

FIG. 1 is a block diagram illustrating an example system for delivering or guiding therapy dosage, in accordance with one or more examples described in this disclosure. FIG. 1 illustrates system 10A that includes patient 12, insulin pump 14, tubing 16, infusion set 18, continuous glucose monitor (CGM) 20, wearable device(s) 22, patient device 24, and cloud 26. Cloud 26 represents a local, wide area or global computing network including one or more processors 28A-28N (“one or more processors 28”).

As illustrated in FIG. 1, system 10A includes cloud 26 that includes one or more processors 28. For example, cloud 26 includes a plurality of network devices (e.g., servers), and the plurality of devices each include one or more processors. One or more processors 28 may be processors of the plurality of network devices, and may be located within a single one of the network devices, or may be distributed across two or more of the network devices. Cloud 26 represents a cloud infrastructure that supports one or more processors 28 on which applications or operations requested by one or more users run. For example, cloud 26 provides cloud computing for using one or more processors 28, to store, manage, and process data on the network devices, rather than by patient device 24 or wearable device(s) 22. One or more processors 28 may share data or resources for performing computations, and may be part of computing servers, web servers, database servers, and the like. One or more processors 28 may be in network devices (e.g., servers) within a datacenter or may be distributed across multiple datacenters. In some cases, the datacenters may be in different geographical locations.

One or more processors 28, as well as other processing circuitry described herein, can include any one or more microprocessors, digital signal processors (DSPs), application specific integrated circuits (ASICs), field programmable gate arrays (FPGAs), or any other equivalent integrated or discrete logic circuitry, as well as any combinations of such components. The functions attributed one or more processors 28, as well as other processing circuitry described herein, herein may be embodied as hardware, firmware, software or any combination thereof.

One or more processors 28 may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. One or more processors 28 may include arithmetic logic units (ALUs), elementary function units (EFUs), digital circuits, analog circuits, and/or programmable cores, formed from programmable circuits. In examples where the operations of one or more processors 28 are performed using software executed by the programmable circuits, memory (e.g., on the servers) accessible by one or more processors 28 may store the object code of the software that one or more processors 28 receive and execute.

Patient 12 may be diabetic (e.g., Type 1 diabetic or Type 2 diabetic), and therefore, the glucose level in patient 12 may be uncontrolled without delivery of supplemental insulin. For example, patient 12 may not produce sufficient insulin to control the glucose level or the amount of insulin that patient 12 produces may not be sufficient due to insulin resistance that patient 12 may have developed.

To receive the supplemental insulin, patient 12 may carry insulin pump 14 that couples to tubing 16 for delivery of insulin into patient 12. Infusion set 18 may connect to the skin of patient 12 and include a cannula to deliver insulin into patient 12. CGM 20 may also be coupled to patient 12 to measure glucose level in patient 12. Insulin pump 14, tubing 16, infusion set 18, and CGM 20 may together form an insulin pump system. One example of the insulin pump system is the MINIMED™ 670G INSULIN PUMP SYSTEM by Medtronic, Inc. However, other examples of insulin pump systems may be used and the example techniques should not be considered limited to the MINIMED™ 670G INSULIN PUMP SYSTEM.

Insulin pump 14 may be a relatively small device that patient 12 can place in different locations. For instance, patient 12 may clip insulin pump 14 to the waistband of pants worn by patient 12. In some examples, to be discreet, patient 12 may place insulin pump 14 in a pocket. In general, insulin pump 14 can be worn in various places and patient 12 may place insulin pump 14 in a location based on the particular clothes patient 12 is wearing.

To deliver insulin, insulin pump 14 includes one or more reservoirs (e.g., two reservoirs). A reservoir may be a plastic cartridge that holds up to N units of insulin (e.g., up to 300 units of insulin) and is locked into insulin pump 14. Insulin pump 14 may be a battery powered device that is powered by replaceable and/or rechargeable batteries.

Tubing 16, sometimes called a catheter, connects on a first end to a reservoir in insulin pump 14 and connects on a second end to infusion set 18. Tubing 16 may carry the insulin from the reservoir of insulin pump 14 to patient 12. Tubing 16 may be flexible, allowing for looping or bends to minimize concern of tubing 16 becoming detached from insulin pump 14 or infusion set 18 or concern from tubing 16 breaking.

Infusion set 18 may include a thin cannula that patient 12 inserts into a layer of fat under the skin (e.g., subcutaneous connection). Infusion set 18 may rest near the stomach of patient 12. The insulin travels from the reservoir of insulin pump 14, through tubing 16, and through the cannula in infusion set 18, and into patient 12. In some examples, patient 12 may utilize an infusion set insertion device. Patient 12 may place infusion set 18 into the infusion set insertion device, and with a push of a button on the infusion set insertion device, the infusion set insertion device may insert the cannula of infusion set 18 into the layer of fat of patient 12, and infusion set 18 may rest on top of the skin of the patient with the cannula inserted into the layer of fat of patient 12.

Sensor 20, which may be herein referred to as a continuous glucose monitor (CGM) 20, may include a sensor that is inserted under the skin of patient 12, such as near the stomach of patient 12 or in the arm of patient 12 (e.g., subcutaneous connection). The sensor of CGM 20 may be configured to measure the interstitial glucose level, which is the glucose found in the fluid between the cells of patient 12. CGM 20 may be configured to continuously or periodically sample the glucose level and a rate of change of the glucose level over time.

In one or more examples, insulin pump 14 and CGM 20, and/or the various components illustrated in FIG. 1, may together form a closed-loop therapy delivery system. For example, patient 12 may set a target glucose level, usually measured in units of milligrams per deciliter, on insulin pump 14. Insulin pump 14 may receive the current glucose level from CGM 20, and in response may increase or decrease the amount of insulin delivered to patient 12. For example, if the current glucose level is higher than the target glucose level, insulin pump 14 may increase the insulin. If the current glucose level is lower than the target glucose level, insulin pump 14 may temporarily cease delivery of the insulin. Insulin pump 14 may be considered as an example of an automated insulin delivery (AID) device. Other examples of AID devices may be possible, and the techniques described in this disclosure may be applicable to other AID devices.

For example, insulin pump 14 and CGM 20 may be configured to operate together to mimic some of the ways in which a healthy pancreas works. Insulin pump 14 may be configured to deliver basal insulin, which is a small amount of insulin released continuously throughout the day. There may be times when glucose levels increase, such as due to eating or some other activity that patient 12 undertakes. Insulin pump 14 may be configured to deliver insulin on demand in association with food intake or to correct an undesirably high glucose level in the bloodstream. In one or more examples, if the glucose level rises above a target level, then insulin pump 14 may increase the insulin dosage amount to address the increase in glucose level. Insulin pump 14 may be configured to compute insulin delivery, and deliver the insulin accordingly. For instance, insulin pump 14 may determine a basal dosage of insulin to deliver continuously, and then determine a bolus dosage of insulin to deliver to reduce glucose level in response to an increase in glucose level due to eating or some other event.

Accordingly, in some examples, CGM 20 may sample glucose level and rate of change in glucose level over time. CGM 20 may output the glucose level to insulin pump 14 (e.g., through a wireless link connection like Bluetooth). Insulin pump 14 may compare the glucose level to a target glucose level (e.g., as set by patient 12 or clinician), and adjust the insulin dosage based on the comparison.

As described above, patient 12 or a clinician may set the target glucose level on insulin pump 14. There may be various ways in which patient 12 or the clinician may set the target glucose level on insulin pump 14. As one example, patient 12 or the clinician may utilize patient device 24 to communicate with insulin pump 14. Examples of patient device 24 include mobile devices, such as smartphones or tablet computers, laptop computers, and the like. In some examples, patient device 24 may be a special programmer or controller for insulin pump 14. Although FIG. 1 illustrates one patient device 24, in some examples, there may be a plurality of patient devices. For instance, system 10A may include a mobile device and a controller, each of which are examples of patient device 24. For ease of description only, the example techniques are described with respect to patient device 24, with the understanding that patient device 24 may be one or more patient devices.

Patient device 24 may also be configured to interface with CGM 20. As one example, patient device 24 may receive information (e.g., glucose level or rate of change of glucose level) directly from CGM 20 (e.g., through a wireless link). As another example, patient device 24 may receive information from CGM 20 through insulin pump 14, where insulin pump 14 relays the information between patient device 24 and CGM 20.

In one or more examples, patient device 24 may display a user interface with which patient 12 or the clinician may control insulin pump 14. For example, patient device 24 may display a screen that allows patient 12 or the clinician to enter the target glucose level. As another example, patient device 24 may display a screen that outputs the current glucose level. In some examples, patient device 24 may output notifications to patient 12, such as notifications if the glucose level is too high or too low, as well as notifications regarding any action that patient 12 needs to take. For example, if the batteries of insulin pump 14 are low on charge, then insulin pump 14 may output a low battery indication to patient device 24, and patient device 24 may in turn output a notification to patient 12 to replace or recharge the batteries.

Controlling insulin pump 14 through patient device 24 is one example, and should not be considered limiting. For example, insulin pump 14 may include a user interface (e.g., pushbuttons) that allow patient 12 or the clinician to set the various glucose levels of insulin pump 14. Also, in some examples, insulin pump 14 itself, or in addition to patient device 24, may be configured to output notifications to patient 12. For instance, if the glucose level is too high or too low, insulin pump 14 may output an audible or haptic output. As another example, if the battery is low, then insulin pump 14 may output a low battery indication on a display of insulin pump 14.

The above describes examples ways in which insulin pump 14 may deliver insulin to patient 12 based on the current glucose levels (e.g., as measured by CGM 20). In some cases, there may be therapeutic gains by proactively delivering insulin to patient 12, rather than reacting to when glucose levels become too high or too low.

The glucose level in patient 12 may increase due to particular user actions. As one example, the glucose level in patient 12 may increase due to patient 12 engaging in an activity like eating or exercising. In some examples, there may be therapeutic gains if it is possible to determine that patient 12 is engaging in the activity, and delivering insulin based on the determination that patient 12 is engaging in the activity.

As illustrated, patient 12 may wear wearable device 22. Examples of wearable device 22 include a smartwatch or a fitness tracker, either of which may, in some examples, be configured to be worn on a patient's wrist or arm. In one or more examples, wearable device 22 includes one or more inertial measurement units, such as a six-axis inertial measurement unit. The six-axis inertial measurement unit may couple a 3-axis accelerometer with a 3-axis gyroscope. Accelerometers measure linear acceleration, while gyroscopes measure rotational motion. Wearable device 22 may be configured to determine one or more movement characteristics of patient 12. Examples of the one or more movement characteristics include values relating to frequency, amplitude, trajectory, position, velocity, acceleration and/or pattern of movement instantaneously or over time. The frequency of movement of the patient's arm may refer to how many times patient 12 repeated a movement within a certain time (e.g., such as frequency of movement back and forth between two positions).

As illustrated in FIG. 1, patient 12 may wear wearable device 22 and/or other wearable devices to avail his/herself of various functionality, for example, in order to output information identifying a recommended food item for patient 12 to eat and/or to identify a particular food item patient 12 is to eat, is eating, or has eaten. In either example, wearable device 22 may generate data indicating (e.g., confirming) consumption of the particular food item by the patient. Another example wearable device may include an Augmented Reality (AR) bracelet.

Patient 12 may wear wearable device 22 on his or her wrist. However, the example techniques are not so limited. Patient 12 may wear wearable device 22 on a finger, forearm, or bicep. In general, patient 12 may wear wearable device 22 anywhere that can be used to determine gestures indicative of eating, such as movement characteristics of the arm.

The manner in which patient 12 is moving his or her arm (i.e., the movement characteristics) may refer to the direction, angle, and orientation of the movement of the arm of patient 12, including values relating to frequency, amplitude, trajectory, position, velocity, acceleration and/or pattern of movement instantaneously or over time. As an example, if patient 12 is eating, then the arm of patient 12 will be oriented in a particular way (e.g., thumb is facing towards the body of patient 12), the angle of movement of the arm will be approximately a 90-degree movement (e.g., starting from plate to mouth), and the direction of movement of the arm will be a path that follows from plate to mouth. The forward/backward, up/down, pitch, roll, yaw measurements from wearable device 22 may be indicative of the manner in which patient 12 is moving his or her arm. Also, patient 12 may have a certain frequency at which patient 12 moves his or her arm or a pattern at which patient 12 moves his or her arm that is more indicative of eating, as compared to other activities, like smoking or vaping, where patient 12 may raise his or her arm to his or her mouth.

Although the above description describes wearable device 22 as being utilized to determine whether patient 12 is eating, wearable device 22 may be configured to detect movements of the arm of patient 12 (e.g., one or more movement characteristics), and the movement characteristics may be utilized to determine an activity undertaken by patient 12. For instance, the movement characteristics detected by wearable device 22 may indicate whether patient 12 is exercising, driving, sleeping, etc. As another example, wearable device 22 may indicate posture of patient 12, which may align with a posture for exercising, driving, sleeping, eating, etc. Another term for movement characteristics may be gesture movements.

Accordingly, wearable device 22 may be configured to detect gesture movements (i.e., movement characteristics of the arm of patient 12) and/or posture, where the gesture and/or posture may be part of various activities (e.g., eating, exercising, driving, sleeping, etc.).

In some examples, wearable device 22 may be configured to determine, based on the detected gestures (e.g., movement characteristics of the arm of patient 12) and/or posture, the particular activity patient 12 is undertaking. For example, wearable device 22 may be configured to determine whether patient 12 is eating, exercising, driving, sleeping, etc. In some examples, wearable device 22 may output information indicative of the movement characteristics of the arm of patient 12 and/or posture of patient 12 to patient device 24, and patient device 24 may be configured to determine the activity patient 12 is undertaking.

Wearable device 22 and/or patient device 24 may be programmed with information that wearable device 22 and/or patient device 24 utilize to determine the particular activity patient 12 is undertaking. For example, patient 12 may undertake various activities throughout the day where the movement characteristics of the arm of patient 12 may be similar to the movement characteristics of the arm of patient 12 for a particular activity, but patient 12 is not undertaking that activity. As one example, patient 12 yawning and cupping his or her mouth may have a similar movement as patient 12 eating. Patient 12 picking up groceries may have similar movement as patient 12 exercising. Also, in some examples, patient 12 may be undertaking a particular activity, but wearable device 22 and/or patient device 24 may fail to determine that patient 12 is undertaking the particular activity.

Accordingly, in one or more examples, wearable device 22 and/or patient device 24 may “learn” to determine whether patient 12 is undertaking a particular activity. However, the computing resources of wearable device 22 and patient device 24 may be insufficient to performing the learning needed to determine whether patient 12 is undertaking a particular activity. It may be possible for the computing resources of wearable device 26 and patient device 24 to be sufficient to perform the learning, but for ease of description only, the following is described with respect to one or more processors 28 in cloud 26.

In some examples, one or more processors 28 may be configured to determine patterns from gesture movements (e.g., as one or more movement characteristics determined by wearable device 22), and configured to determine a particular activity patient 12 is undertaking. One or more processors 28 may provide responsive real-time cloud services that can, on a responsive real-time basis, determine the activity patient 12 is undertaking, and in some examples, provide recommended therapy (e.g., insulin dosage amount). Cloud 26 and patient device 24 may communicate via WiFi or through a carrier network.

For example, as described above, in some examples, wearable device 22 and/or patient device 24 may be configured to determine that patient 12 is undertaking an activity. However, in some examples, patient device 24 may output information indicative of the movement characteristics of movement of the arm of patient 12 to cloud 26, and possibly with other contextual information like location or time of day. One or more processors 28 of cloud 26 may then determine the activity patient 12 is undertaking. Insulin pump 14 may then deliver insulin based on the determined activity of patient 12.

The above describes arm movement as a factor in determining whether patient 12 is engaging in an activity. However, there may be various other factors that can be used separately or in combination with arm movement to determine whether patient 12 is engaging in an activity. As one example, patient 12 may engage in the activity at a regular time intervals. As another example, patient 12 may engage in the activity at certain locations. In the initial learning phase, when patient 12 enters that he or she is engaging in the activity (e.g., through patient device 24), patient device 24 may output information about the time of day and the location of patient 12. For example, patient device 24 may be equipped with a positioning device, such as global positioning system (GPS) unit, and patient device 24 may output location information determined by the GPS unit. There may be other ways to determine location such as based on Wi-Fi connection and/or access to 4G/5G LTE, or some other form of access, such as based on telecom database tracking device location of patient device 24. Time of day and location are two examples of contextual information that can be used to determine whether patient 12 is engaging in the activity.

However, there may be other examples of contextual information for patient 12 such as sleep pattern, body temperature, stress level (e.g., based on pulse and respiration), heart rate, etc. In general, there may be various biometric sensors (e.g., to measure temperature, pulse/heart rate, breathing rate, etc.), which may be part of wearable device 22 or may be separate sensors. In some examples, the biometric sensors may be part of CGM 20.

Other examples of contextual information for patient 12 include possible or recommended food items (e.g., solid meat or vegetables and liquids beverages) for consumption. Eating (e.g., during meal events) is an activity engaged in by patient 12 and identifying which food items are being consumed while eating may serve an important role in diabetes insulin delivery for that patient 12. In various aspects including nutrition and volume, two food items may differ by small or large margins. When a chicken drumstick is compared to an apple, for instance, each provides different nutrients and contributes a different amount to patient 12's body mass. Techniques described herein may be directed towards identifying a particular food item from various information including the contextual information described herein and as part of identifying the food item, some techniques may be configured to determine supporting information (e.g., therapy information for the patient and other food item information such as nutrient and volume information) for the diabetes insulin delivery for patient 12.

The contextual information for patient 12 may include conditional information. For example, patient 12 may eat every 3 hours, but the exact times of when patient 12 eats may be different. In another example, patient 12 may eat a meal every time he is at particular location (e.g., a restaurant, food court, cafeteria). In some examples, the conditional information may be a determination of whether patient 12 has eaten and if a certain amount of time (e.g., 3 hours) has passed since patient 12 ate. In general, any information that establishes a pattern of behavior may be utilized to determine whether patient 12 is engaging in a particular activity (e.g., eating a particular food item such as a steak, an apple, or a tomato).

One or more processors 28 may utilize artificial intelligence, such as machine-learning or other data analytics techniques, based on the information determined by and/or collected by wearable device 22 and patient device 24 to determine whether patient 12 is engaging in the activity. As one example, during the initial learning phase, one or more processors 28 may utilize neural network techniques. For example, one or more processors 28 may receive training data from patient 12 that is used to train a classifier module executing on one or more processors 28. As described above, one or more processors 28 may receive the training data based on patient confirmation when patient device 24 and/or wearable device 22 determine, based on manner and frequency of movement of the arm of patient 12, that patient 12 is engaging in the activity (e.g., a gesture that aligns with movement of arm for eating). One or more processors 28 may generate and store a labeled data record that includes the features related to the movement, along with other contextual features, such as time of day or location. One or more processors 28 may train the classifier on a labeled dataset that includes multiple labeled data records, and one or more processors 28 may use the trained classifier model to more accurately detect the start of a food intake event.

Other examples that may be used for neural networks include behavior patterns. For example, patient 12 may only eat a particular food after exercising, and always eats that particular food after exercising. Patient 12 may eat at a particular time and/or place. Although described with respect to eating, there may be various conditions that together indicate a pattern in behavior of patient 12 for different activities.

As another example, one or more processors 28 may utilize k-means clustering techniques to determine whether patient 12 is engaging in an activity. For example, during the initial learning phase one or more processors 28 may receive different types of contextual information and form clusters, where each cluster represents a behavior of patient 12 (e.g., eating, sleeping, walking, exercising, etc.). For example, patient 12 may enter information (e.g., into patient device 24) indicating that he or she is walking. One or more processors 28 may utilize all the contextual information received when patient 12 is walking to form a first cluster associated with walking. Patient 12 may enter information (e.g., into patient device 24) indicating that he or she is eating and which food item(s) he or she is eating. One or more processors 28 may utilize all the contextual information received when patient 12 is eating to form a second cluster associated with eating, and so on. Then, based on received contextual information, one or more processors 28 may determine which cluster aligns with the contextual information, and determine the activity patient 12 is undertaking. As described in more detail, the type of activity, and a prediction of when the activity will occur, may be utilized to determine when to delivery insulin therapy. There may be other examples of machine learning, and the example techniques are limited to any particular machine learning technique.

There may be various other ways in which one or more processors 28 may determine the activity patient 12 is undertaking. This disclosure provides some example techniques for determining the activity patient 12 is undertaking, but the example techniques should not be considered limiting.

During the initial learning phase, patient 12 may also enter information about the activity that patient 12 is undertaking. For example, with eating, patient 12 may enter information indicating what patient 12 is eating and/or how many carbohydrates there are in the food that patient 12 is eating. As one example, at 9:00 every morning, patient 12 may enter that he or she is having a bagel or enter that the patient 12 is consuming 48 grams of carbohydrates.

In some examples, one or more processors 28 may be configured to determine an amount of insulin (e.g., bolus dosage) to deliver to patient 12. As one example, memory accessible by one or more processors 28 may store patient parameters of patient 12 (e.g., weight, height, etc.). The memory may also store a look-up table that indicates an amount of bolus dosage that is to be delivered for different patient parameters and different types of foods. One or more processors 28 may access the memory and based on the type of food patient 12 is eating and patient parameters may determine the amount of bolus dosage that patient 12 is to receive.

As another example, one or more processors 28 may be configured to utilize a “digital twin” of patient 12 to determine an amount of insulin patient 12 is to receive in a single dose. Depending on which time of day patient 12 is scheduled to receive the determined amount of insulin, the insulin dose may be considered a basal dosage or a bolus dosage. A digital twin may be a digital replica or model of patient 12. The digital twin may be software executing on one or more processors 28. The digital twin may receive, as input, information about what patient 12 ate and/or is eating. Because the digital twin is a digital replica of patient 12, the output from the digital twin may be information about what the glucose level of patient 12 may be after eating, as well as a recommendation of how much bolus dosage or basal dosage to deliver to patient 12 to control the increase of the glucose level. Accordingly, a digital twin of patient 12 allows analysis of real-time data (e.g., what patient 12 is eating), impact on patient 12 (e.g., how much change there will be in the glucose level), and/or therapy recommendations (e.g., how much insulin to provide).

For example, the digital twin may indicate what the correct dose should have been for a meal that patient 12 ate in the past. In one or more examples, patient 12 may enter information indicative of food patient 12 ate and one or more processors 28 may receive information about glucose levels. Utilizing information indicative of food that patient 12 ate and glucose levels, one or more processors 28 may utilize the digital twin to determine what the insulin dose should have been (e.g., based on how the digital twin models how the food will affect the patient's glucose level). Then, at a subsequent time when patient 12 is predicted to eat the same meal, one or more processors 28 may determine what the insulin dose should be based on insulin dose amount that the digital twin had previously determined.

As described herein, a kit of baseline edible consumables in the form of solid or liquid foods (or some combination therein), in combination with the digital twin models, may be used to calibrate patient 12's therapy to patient 12's physiology. By consuming substantially the same nutrients over a number of meals or days, one or more processors 28 may determine parameters to program into the digital twin models in order to accurate determine bolus or basal dosage amounts and/or a schedule that patient 12's is to be administered insulin treatments. In some examples, cloud 26 is combined with a kit of baseline edible solid bars and one or more processors 28 is configured to adjust the digital twin model's parameters over a number of iterations until the digital twin algorithm is sufficiently trained. In some examples, one or more processors 28 may adjust the parameters within three days.

Accordingly, in one or more examples, one or more processors 28 may utilize information about the movement characteristics of movement of arm, eating pace, quantity of food consumption, food content, etc., while also tracking other contextual information. Examples of the contextual information include location information, time of day, wake up time, amount of time since last eaten, calendar event, information about person patient 12 may be meeting, etc. One or more processors 28 may identify patterns and correlations between all these various factors to determine an activity patient 12 undertaking, like eating, walking, sleeping, driving, etc.

After the initial learning phase, one or more processors 28 may automatically, and with minimal input from patient 12, determine that patient 12 is undertaking a particular activity, and determine an amount of bolus insulin to deliver based on the determination. One or more processors 28 may output the recommendation of the amount of insulin to deliver to patient device 24. Patient device 24, may then in turn, control insulin pump 14 to deliver the determined amount of insulin. As one example, patient device 24 may output to insulin pump 14 the amount of insulin to deliver. As another example, patient device 24 may output a target glucose level, and insulin pump 14 may deliver the insulin to achieve the target glucose level. In some examples, it may be possible for one or more processors 28 to output to patient device 24 information indicative of the target glucose level, and patient device 24 may output that information to insulin pump 16. All of these examples may be considered as examples of one or more processors 28 determining an amount of insulin to deliver to patient 12.

The above describes example ways in which to determine if patient 12 is undertaking an activity, determining an amount of insulin to deliver, and causing the amount of insulin to be delivered. The example techniques may require little to no intervention from patient 12. In this manner, there is an increase in likelihood that patient 12 will receive the correct amount of dosage of insulin at the right time, and decrease in likelihood of human error causing issues (e.g., patient 12 forgetting to log meals, forgetting to take insulin, or taking insulin but forgetting to have taken insulin).

Although the above describes proactive determination of patient 12 eating and delivering insulin accordingly, the example techniques are not so limited. The example techniques may be utilized for proactively determining an activity that patient 12 is undertaking (e.g., eating, exercising, sleeping, driving, etc.). Insulin pump 14 may then deliver insulin based on the determination of the type of activity patient 12 is undertaking.

While the above example techniques may be beneficial in patient 12 receiving an effective amount of insulin at the right time, this disclosure describes example techniques to further proactively control delivery of insulin to patient 12.

As illustrated in FIG. 1, cloud 26 maintains structured data, such as food data 27, in data store(s) including various types of food-related information. In the food-related information for a particular food item, a number of informational attributes may be arranged in accordance with a pre-determined structure as described herein. A food item, in general, refers to any edible volume that a person consumes for some physiological benefit. In the present disclosure, patient 12 is a diabetes patient who derives food energy from various food items where each food item is composed of a different combination of carbohydrates, fats, proteins, organic acids, polyols, and/or ethanol. Any given food item may be represented by their nutrient composition. Food data 27 may include, as an example representation, food item profiles of which each profile defines a corresponding food item as a combination of carbohydrates, fats, proteins, organic acids, polyols, and/or ethanol. Food item profiles, in general, facilitate the identification of specific food items and the differentiation between different food items by their nutrient composition.

An example food item profile's structure is configured to arrange, into a set of attributes, a food item's unique nutrient composition, including macronutrient and/or micronutrient information. A particular food item's set of attributes may include (well-known or pre-determined) nutritional facts regarding that particular food item's composition of carbohydrates, fats, proteins, organic acids, polyols, and/or ethanol. Other example attributes further describe the particular food item with information indicating the particular food item's mass and/or volume (e.g., in terms of serving size(s)) and including information describing the particular food item's shape and/or size. As described herein, a machine learning model may use the food item profiles as representations that are suitable for machine learning techniques (e.g., and for predicting patient 12's nutrition state).

For a variety of reasons, some food items may have an incomplete or undetermined nutrient composition where some nutritional facts are unknown or are uncertain. For certain food items, a nutritional fact may be an approximation that is derived from similar food items (e.g., food items of a same food type), but that approximation may not accurately describe a food item's actual nutritional composition (e.g., by volume). If the food item's volume is unknown, some nutritional facts may not be determinable. Any of these inaccuracies may inhibit an accurate prediction by the machine learning model. Some example profiles identify which attribute(s) may be unknown (e.g., at a present time). Even though a given food item consists of carbohydrates, fats, proteins, water, vitamins, and minerals, a substantial number of foods items are primarily composed of carbohydrates, fats, proteins, and water; for at least this reason, their food item profiles may not include attributes describing quantities of vitamins and minerals.

As described herein, with respect to a specific food item such as an apple or a generic food type such as fruit, one or more processors 28 may retrieve from food data 27 relevant information for determining, for example, a single gala apple's nutritional value and health impact to patient 12 (if consumed). One or more processors 28 may identify attributes defining the apple's (e.g., known or pre-determined) nutritional facts. By “nutritional facts”, the present disclosure refers to the apple's chemical composition of specific molecules that compose at least a substantial portion of the apple. These chemical molecules may be nutrients that are either known (e.g., publicly) or pre-determined (e.g., via testing or using instruments to take measurements). In addition to identifying which specific nutrients are present in the apple (e.g., by their substance names and/or formulas), one or more processors 28 may retrieve from food data 27 other information describing these specific nutrients, for example, in terms of quality and/or quantity. To illustrate by way of example, the above-mentioned apple may include carbohydrates (e.g., sugars, alcohols, and/or the like), lipids (e.g., fats, sterols, and/or the like), salts (e.g., sodium ions, electrolytes, and/or the like), proteins (e.g., polymer chain of amino acids), water molecules, and/or other molecular classes. To specify by way of further example, one or more processors 28 may determine that the above-mentioned apple comprises, in respective non-trivial percentages, sugar molecules (e.g., fructose, sucrose, and/or glucose), fat molecules (e.g., Omega-3, 6, and/or 9 fatty acids), sterol molecules (e.g., Cholesterol), salt molecules (e.g., sodium), and so forth.

Based on information describing a nutrient composition for a particular food item patient 12 is to consume, is consuming, or has consumed within a (recent) time period, one or more processors 28 may determine whether patient 12's biomarker (e.g., glucose) level is likely to change (e.g., by a predictable amount) and if so, determine whether that changed biomarker (e.g., glucose) level affects patient 12's current or future nutrition state. In some examples, one or more processors 28 may predict that patient 12's glucose level changes to an extent that it could negatively affects patient 12's health (e.g., by disrupting therapy delivery). One or more processors 28 may retrieve from model 29 a trained machine learning model for predicting patient 12's biomarker level after patient 12 consumes the particular food item. The trained machine learning model stored in model 29 may include a mathematical function, a prediction/classification algorithm, and/or a probability distribution where each of the function, algorithm and/or distribution has learnt, from a training process, how to accurately predict patient 12's biomarker level after patient 12 consumes the particular food item; such predictions are determined from on input (feature) data, which may (in part) be based on information corresponding to the particular food item's nutrient composition.

It should be noted that food data 27 may be configured to define a food item's molecular composition at multiple levels of granularity such that, at one level of granularity, a fine-grained composition decouples specific nutrients in that food item; to illustrate by way of the above-mentioned apple, an example fine-grained composition may specify quantities (e.g., as respective percentages) for different sugar molecule types (e.g., fructose, sucrose, and/or glucose) for that apple. On the other hand, food data 27 may specify an approximate quantity (e.g., as a cumulative percentage or sub-total) for the apple's entire sugar molecule composition. For this level of granularity in food data 27, the cumulative percentage of any given molecular class may be described as an approximation whose accuracy depends on a number of factors. If given a precise volume estimate for the apple (or any food item in this regard), one or more processors 28 may determine an accurate percentage of that molecule class or specific molecule type.

Utilizing known scientific principals and/or published materials, one or more processors 28 may define the above apple with known or pre-determined measurement(s) in terms of a standardized quantity/quality, such as in a number of calories. Food data 27, in a nutritional context, may describe at least one attribute of the above-mentioned apple (e.g., in terms of a quality or a quantity) using a scientific unit of energy (e.g., nutritional or food energy). The calorie, one example unit of energy, is defined as the amount of heat needed to raise a quantity of water by one degree of temperature. Calories (e.g., kilocalories (kcal)) and kilojoule (kJ), another example SI unit of energy (e.g., nutritional or food energy), are commonly used measures of a chemical energy that humans derive from their food such as the above apple. Food data 27 is not limited to quantities (e.g., counts) of calories (e.g., per gram or serving size) and may include additional or alternative scientific measurements.

For the above single apple, food data 27 may define a standardized calorie count where a certain mass is assumed such that each apple's caloric count is the same and actual caloric differences between individual apples are abstracted out for having a trivial influence on the caloric count. In addition or as an alternative, food data 27 may define a caloric ratio per specific unit of mass for the above single apple (e.g., an average calorie count per gram or serving size). For specific nutrients, food data 27 may define a caloric ratio per specific unit of mass, such as lipids (e.g., fats) which have 9 kilocalories per gram (kcal/g), both carbohydrates (e.g., sugars and fibers) and proteins (e.g., amino acid polymers) contain approximately 4 kcal/g, and alcohol (e.g., ethanol) in food/beverage contains 7 kcal/g.

Model 29, in general, represents dedicated storage space for storing information to facilitate implementing, training, and then, applying trained machine learning models to determine various types of information. In cloud 26, one or more processors 28 provide a number of mechanisms for representing machine learning models as computer data (e.g., a data structure) for storage in model 29. Via an interface to cloud 26, an external device, such as patient device 24, may access resources and services corresponding to specific trained machine learning models. For example, patient device 24 may submit a request for access to a downloadable copy of a trained model; in turn, one or more processors 28 may return a response to patient 12's request with one or more files storing data for the requested trained model's representation. In another example, patient device 24 may submit a request for access to a trained model via a cloud service; in turn, one or more processors 28 may return a response acknowledging initiation of a process-to-process session. In other examples, patient device 24 may have direct access to model representations stored in model 29 and control over dedicated software programs to read a model's architecture, identify the model's data components (e.g., input features, parameters, hyperparameters, filters including kernels, and/or the like), and perform some computerized task.

For each machine learning model in general, model 29 includes information (e.g., software code) implementing an example machine learning technique (e.g., an evaluation technique configured to analyze input feature data and then, generate prediction data as output). It should be noted that the evaluation technique described herein may be any mathematical function or probability distribution where at least one variable can be modeled and further includes different combinations of mathematical functions and/or probability distributions such as in a computer algorithm (e.g., a prediction algorithm).

Another example machine learning technique (e.g., a training technique) may be configured to generate a trained machine learning model and an applicable evaluation technique for that model. As described herein, the training technique may involve an algorithm (e.g., a learning algorithm) for finding at least one combination of model components (e.g., values) that results in an acceptable (e.g., minimally accurate) evaluation technique. For one example trained model, the training technique may produce an evaluation technique configured to generate, with a high degree of precision, data for patient 12's predicted nutrition state, including (accurate or even optimal) estimations of patient 12's biomarker level(s) at either a present point-in-time or within a short time period. The evaluation technique may be configured to generate likely biomarker level estimates at one or more past points in time.

Any of the above machine learning techniques may be codified, for example, as software code in either a dedicated software program for patient device 24 or a compute node for the cloud computing service, and when executed, processing circuitry in either patient device 24 or cloud 26 may apply the technique through a running process (e.g., an operating system process to run in user space or kernel space). For example, one or more processors 28 may

To prepare food data 27 for evaluation by the trained machine learning model, one or more processors 28 may arrange various food item information into datasets of input features according to one example. As described herein for at least one example feature dataset, one or more processors 28 may utilize one or more attributes of food item information to define each unique food item in which each set of attributes may be formatted into a structure—which may herein be referred to as a food item profile. Another example feature dataset may define each food item with the same set of attributes and further include one or more additional features. One or more processors 28 apply the trained machine learning model to one or more feature datasets and then, render a prediction regarding patient 12's nutrition state, food data 27

In general, cloud 26 may be configured to provide cloud clients, such as patient 12 and patient device 24, access to various data and computing services involving one or more machine learning models in model 29. Cloud 26 may operate an example environment on which one or more processors 28 may instantiate, as a software object, a trained machine learning model stored in model 29 and then, execute, via one or more processors 28, executable instructions configured to run a machine learning technique on behalf of the cloud clients.

Cloud 26 may host an example computing service with machine learning capabilities to the benefit of patient 12, for example, by providing patient 12 with information (e.g., a prediction) describing patient 12's nutrition state for a specific time period. To run the example computing service, one or more processors 28 may employ the trained machine learning model to predict a current nutrition state in response to patient 12's recent consumption of a particular food item and/or a future nutrition state for patient 12 if that patient consumes a particular food item. The above example computing service may invoke an appropriate machine learning technique (e.g., evaluation technique) to apply the trained machine learning model to a set of attributes in the particular food item's profile and then, generate a predicted patient nutrition state.

A person's “nutrition state” as described herein generally refers to the person's physiology as defined by one or more biomarkers (e.g., one or more biomarker levels). The present disclosure may refer to “nutrition state” with alternative phrasings, such as “biomarker state” or “physiological” state. Patient 12's nutrition state may change based on his or her nutritional intake with respect to each nutrient's quality/quantity. Cloud 26 and the computing service may define the nutrition state for any given person as a combination of different biomarker estimates and/or measurements and (possibly) other information. One or more sensor devices may sense a biomarker and then, measure the biomarker's (current) level. Based on that biomarker measurement and a nutritional composition of one or more food items that patient 12 (currently) is consuming, (recently) has consumed, or (presumably) is to consume in the imminent future, one or more processors 28 may determine whether patient 12's current biomarker level is likely to change upon consumption of the one or more food item and if so, determine by what amount the person's current biomarker level is likely to increase or decrease.

When configured with a machine learning model configured to predict biomarker levels for a diabetes patient, such as patient 12, one or more processors 28 may generate that patient's predicted nutrition state to indicate one or more estimated and/or sensed biomarker levels in the patient's imminent future. For the above nutrition state, one or more processors 28 may store an estimate and/or a measurement for each biomarker level and correlate each estimated biomarker level and/or measured biomarker with at least one particular point-in-time. One or more processors 28 may generate the nutrition state to indicate any biomarker level currently or imminently likely to be at an unhealthy level (in general) or a risky level (for a specific ailment). If any biomarker level is unhealthy or risky, one or more processors 28 generate, for output, information (e.g., an alert) indicating one or more potential health issues likely to result from the unhealthy or risky biomarker level. To illustrate by way of one example biomarker, one or more processors 28 may generate patient 12's nutrition state indicating a glucose level that patient 12 is most likely to have in their immediate future after consuming a particular food item. One or more processors 28 may estimate the likely increase or decrease in patient 12's glucose level after consumption due to the fact that intaking the particular food item's nutritional composition may result in a physiological response by patient 12 that is likely to change patient 12's glucose level.

The above machine learning model may undergo a training process to calibrate estimations of patient 12's biomarker levels to patient's actual biomarker levels after patient 12 consumes one or more food items. As described herein, the training process generally refers to the training technique described herein and to a computing process operative to execute the training technique on the above model. To effectuate accurate estimations of patient 12's biomarker levels, the training process may modify model components (e.g., weights and other parameters) to iteratively calibrate the model to patient 12's physiological responses to food items. Depending on objective, one or more processors 28 may run the training process until the model is sufficiently trained to estimate biomarker levels for a specific demographic group or sub-group (e.g., for diabetes patients). In some examples, one or more processors 28 may run the training process and calibrate the above model to only patient 12's physiology.

At any iteration, to assess the above machine learning model's training thus far, one or more processors 28 may compare at least one estimated biomarker level with at least one actual (e.g., measured) biomarker level for a same time period. Based on such a comparison, one or more processors 28 may determine the model's accuracy in estimating the patient's biomarker levels based on a food item's nutritional composition. One or more processors 28 may apply a suitable accuracy metric configured to assess the model's accuracy (e.g., defined as the model's estimation error) and if an accuracy metric value exceeds a threshold value (e.g., 95%), one or more processors 28 may consider the model to be sufficiently trained and therefore, ready for deployment via the computing service in cloud 26 and/or the patient's device, such as patient device 24. Further improvements to the model may not be efficient when metric values plateau during training. If, on the other hand, the metric value does not exceed the threshold value, one or more processors 28 may have the model undergo further training. Hence, the metric allows one or more processors 28 to distinguish models that require calibration and models that are sufficiently trained and do not require further calibration.

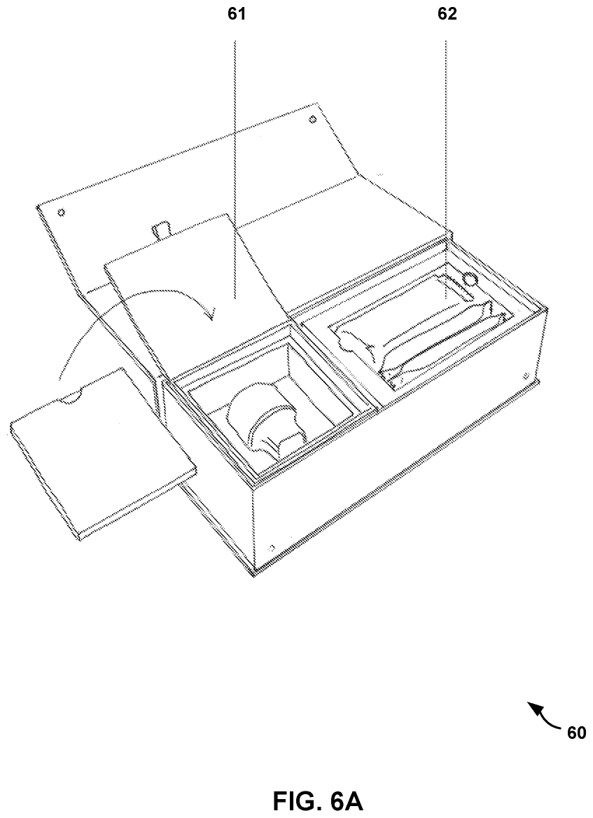

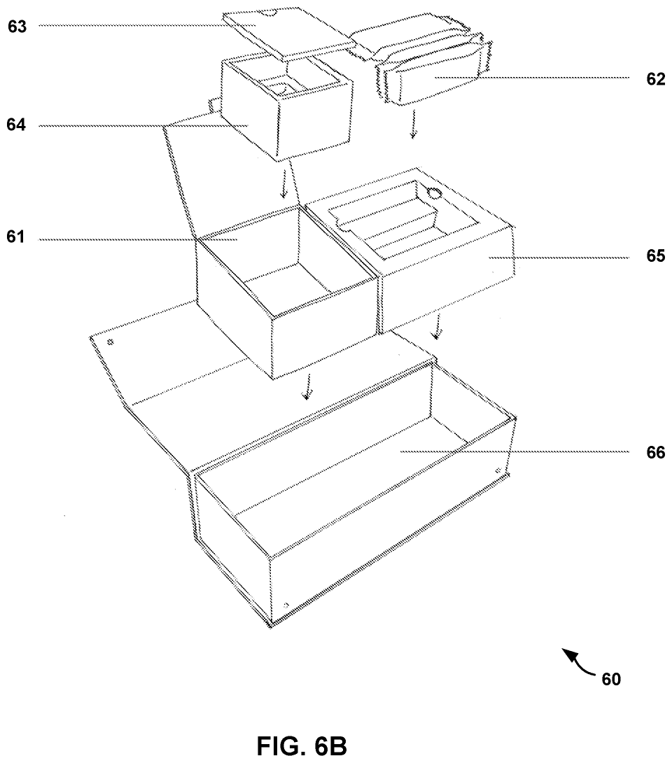

As described herein, training the above model until satisfaction of at least one criterion (e.g., metric threshold value) may ensure that model's effectiveness in managing the patient's biomarker levels. Calibrating the above model to make predictions specifically for patient 12 may require further training but even with additional iterations, the above model may not achieve a pre-defined desired level of accuracy. To enable such calibration, a number of pre-determined baseline food items (e.g., in a kit such as the one illustrated by FIGS. 6A and 6B) may facilitate training of the above model to make personalized predictions for patient 12. One or more processors 28 may avail pre-determined nutrition information for the baseline food items to accurately estimate patient 12's biomarker levels in response to patient 12 consuming other food item (e.g., in accordance with a specific schedule).

When given a trained machine learning model configured to accurately predict biomarker levels for persons in general, diabetes patients in particular, or patient 12 specifically, one or more processors 28 may apply that trained machine learning model to input features in one or more food item profiles and generate a predicted patient nutrition state for each of the one or more food item profiles. The one or more food items may represent an inventory of food items patient 12 may access for consumption; unlike the pre-determined baseline food items for facilitating the model's training/calibration, some inventory food items may have one or more unknown nutritional facts but may be of a known food type and/or have known ingredients. While food data 27 may store a pre-defined profile with known nutritional facts as attributes for one example food item, food data 27 may store determined or standardized nutritional facts as attributes for another example food item. Food producers typically publish nutritional facts for their food item products, and food data 27 may incorporate those nutritional facts into food item profiles. If a particular food item has unknown nutrition attributes or nutrition attributes that have yet to be determined, there are a number of ways (e.g., formulas) to determine a nutrition attribute's value with a high degree of precision. Besides using mathematical techniques such as extrapolation/interpolation, there may be specific formulas for combining nutritional facts of the particular food item's ingredients. In other examples, the particular food item may be similar (e.g., in mass and/or volume) to other food items of a same food type, and food data 27 may store a standardized set of nutrition attributes for the food items of that food type. One or more processors 28 may further employ a number of devices to compute values for the particular food item's nutrition attributes.

Based on each food item's predicted patient nutrition state, one or more processors 28 may select at least one food item to recommend for consumption by patient 12. One or more processors 28 may identify, from amongst the one or more food items, the at least one selected food item to recommend based on a comparison between the predicted patient nutrition state and a desired patient nutrition state for persons in general, diabetes patients in particular, or patient 12 specifically. As described herein, the predicted patient nutrition state refers to a point in time after patient 12 consumes a corresponding food item; for example, the predicted patient nutrition state may indicate at least one estimated biomarker level expected to be within at least one desired range if patient 12 consumes the one or more selected food item. Depending on recommendation criteria, one or more processors 28 may select, for patient 12's recommendation, any food item likely to cause the above predicted patient nutrition state. In a further example, one or more processors 28 may select, for patient 12's recommendation, any food item likely to result in a predicted patient nutrition state having at least one estimated biomarker level expected to be within at least one desired range, for example, assuming a delivery device such as insulin pump 14 executes therapy information and the patient consumes the selected food item. Delivery devices, as described herein, are configured to execute the therapy information to direct therapy delivery to the patient, for example, in accordance with a treatment schedule for administering specific treatment amounts for a therapy time period. One or more processors 28 may generate output data, for display, indicating selected food item(s) to recommend to patient 12.

One or more processors 28 of cloud 26 may deploy a client application to run in patient device 24 in support of the above-mentioned therapy delivery or, as an alternative, to run as a stand-alone application capable of running a machine learning technique (e.g., an evaluation technique). Cloud 26 may configure a communication link to facilitate communications between one or more processors 28 and the client application running in patient device 24. In some examples, one or more processors 28 is to communicate various data retrieved from portions of food data 27 and/or model 29 and instructions for implementing the evaluation technique. As described herein, the evaluation technique may involve applying a trained model retrieved from model 29 on a food item profile retrieved from food data 27. Based on a predicted patient 12 nutrition state resulting from such an application, one or more processors 28 may direct the client application to either recommend to patient 12 that he/she consume the particular food item or discourage the particular food item's consumption.

With or without assistance from cloud 26, the above client application may perform a machine learning technique in a manner that provides at least some benefit to patient 12. A running operating system (e.g., kernel) process within patient device 24 may execute a software program for the client application, which (in turn) instantiates a running process to implement an evaluation technique, a training technique, or another machine learning technique described herein. To effectuate the training technique, the client application may execute a training process to (sufficiently) train a machine learning model for accurately predicting patient 12's current nutrition state given patient 12's past (e.g., recent) diet or patient 12's future nutrition state given patient 12's cotemporaneous or imminent consumption of one or more food items; and if the running training process determines that the model's predictions are sufficiently accurate (e.g., in satisfaction of an accuracy metric threshold value), the client application may deploy the now trained model for use in controlling patient 12's diet. As described herein, a controlled diet may benefit patient 12 with improved diabetes management and better health overall. To effectuate the evaluation technique, the client application may execute an evaluation process configured to (ultimately) apply the trained model to input features provided by food data 27 or local data and render a prediction that provides some benefit or advantage to patient 12.

To illustrate by way of example, patient 12 may submit requests (e.g., service request) to either cloud 26 or the above-mentioned client application where each request is a query concerning patient 12's food intake. In turn, cloud 26 and/or the client application perform task(s) in accordance with the request and then, provide response data in completion of each query submitted by patient 12. As one example, patient 12 may submit a request to seek a recommendation as which food item to each next or in near future. Such a request does not assume patient 12 plans on consuming any particular food item. If patient 12 has a particular food item in mind as a next food item to consume, patient 12 may submit a request desiring an evaluation of the particular food item for possible consumption. Such a request may prompt one or more processors 28 or the client application to generate a predicted patient nutrition state that is likely to result if patient 12 actually consumes the food item. In response to the above request, one or more processors 28 and/or the client application may generate (for display) output indicating an appropriate response for patient 12 to avail. Patient 12 may decide whether or not to consume the particular food item in view of the predicted nutrition state, thus leveraging a machine learning capability of cloud 26 and/or the client application in making good (e.g., healthy) dietary choices, especially in consideration of patient 12's diabetes.

Patient 12 (or patient 12's doctor or caregiver) may submit, in an example request, a specific query regarding patient 12's current/future nutrition state (e.g., including any one or more biomarker levels); and as described herein, one example query requests, from the trained machine learning model, a recommendation for an appropriate food item to consume (e.g., within a certain time period) given patient 12's current/future nutrition state and (possibly) other additional factors to considers. The application process running in cloud 26 may select a particular food item to recommend based on that food item's likelihood (e.g., probability) of placing patient 12's current/future nutrition state (e.g., biomarker level(s)) at or near a desired nutrition state (e.g., desired biomarker level(s)). Patient 12 may define the desired nutrition state in terms of established threshold biomarker level(s). Patient 12 may set a desired biomarker level at a static value. One example desired biomarker level may be an optimal or near-optimal biomarker level for patient 12's physiology. As another example, patient 12 may configure the desired biomarker levels in accordance with patient 12's physician's instructions. Patient 12's doctor's may prescribe a treatment (e.g., diabetes treatment) and a therapy for applying the treatment and set forth instructions for patient 12 to follow including desired biomarker levels to maintain in order for patient 12 to achieve a healthy nutrition state. Alternatively, patient 12 may configure threshold biomarker level(s) to be dynamic, for example, adjusting in view of patient 12's inconsistent lifestyle (e.g., diet and physical activities such as exercising) and/or patient 12's overall health. Patient 12's doctor may passively adjust patient 12's desired biomarker levels as described herein.

It should be noted that patient 12 is not required to issue any of the above request, and for example, one or more processors 28 and/or the client application may assume patient 12's desire for healthy dietary recommendations. The present disclosure may assume such a desire and provide such recommendations passively (e.g., when patient 12 is eating) and/or periodically. The present disclosure describes one or more additional factors that may be incorporated into the machine learning evaluation and recommendation techniques described herein.

As described herein, patient 12 may be provided with baseline food items for use in training a personalized machine learning model. FIG. 6A and FIG. 6B are illustrations of a packaging structure for a kit consisting of bars as examples of the baseline food items; in other kits, the baseline food items may include beverages or a combination of bars and beverages. An example kit may accompany patient device 24 and, via communication link with cloud 26, may be communicatively coupled to one or more processors 28. In some examples, a set of baseline foods may be included within the kit. Food data 27 stores for each baseline food item various information including predetermined nutritional facts/information for each of the baseline food items by cloud 26. When patient 12 consumes a baseline food item (e.g., in accordance with a pre-determined schedule) and the personalized machine learning model is applied to the baseline food item's nutritional facts, one or more processors 28 in cloud 26 and/or the client application in patient device 24 may generate a predicted patient nutrition state that indicates (in part) one or more estimated biomarker levels. Based on such a prediction, one or more processors 28 and/or the client application continues the personalized model's training by comparing the one or more estimated biomarker levels to actual biomarker level measurements. Such a comparison may be indicative of a degree to which the personalized model accurately predicts patient 12's biomarker levels. One or more processors 28 and/or the client application in patient device 24 may employ a number of applicable metrics to determine the personalized model's degree or level of accuracy and if that determination does not satisfy one or more criteria, the training of the personalized model resumes. For example, if an example metric generates a value indicative of the personalized model's accuracy and that generated value is below a threshold value, one or more processors 28 and/or the client application in patient device 24 resumes training the personalized model's for a next baseline food item in the kit. In another example, the kit may include a suitable number of baseline food items to sufficiently train the personalized model and thus, patient 12's consumption of a last baseline food item signals the conclusion of the training technique.

Once fully trained, one or more processors 28 and/or the client application in patient device 24 may output a trained personalized machine learning model for patient 12 to avail for their benefit. Such a model may be configured specifically for patient 12's physiology and thus, suitable to manage patient 12's diabetes. As described herein, the (trained) personalized machine learning model may be used to support patient 12's diabetes therapy by accurately predicting patient 12's expected nutrition state in response to patient 12 consuming a particular food item. Based on an estimated biomarker level that is likely to result from consumption of the particular food item, patient 12's current therapy may be adapted to effectively account for the particular food item's nutrition composition. The above description assumes patient 12 consumed the particular food item already; however, if patient 12 has not yet consumed the particular food item, the above accurate prediction may prompt one or more processors 28 or patient device 24 to alert patient 12 of the expected biomarker level increase or decrease in an effort to prevent consumption and thus, the estimated biomarker level that is likely to result from patient 12 intaking the particular food item's nutrition composition.

A delivery device (e.g., insulin pump 14) coupled to patient device 24 may incorporate such a prediction into patient 12's therapy delivery, for example, by determining an appropriate treatment amount, treatment type, and/or treatment schedule for patient 12's diabetes therapy. One or more processors 28 and/or the client application in patient device 24 may apply the above trained personalized machine learning model to determine whether a particular food item's nutrient composition is likely to increase or decrease patient 12's glucose level by a non-trivial amount. For example, for substantial percentage of patients but not all patients, orange juice significantly increases glucose levels while red wine decreases glucose levels. The above trained personalized machine learning model accounts for patient 12's personal physiological response with respect to red wine and/or orange juice. The above trained personalized machine learning model may also distinguish red wine and white wine in view of their respective impacts on patient 12's glucose level.

If, in one example, patient 12's predicted nutrition state indicates that patient 12's current (e.g., measured) glucose level is expected to increase by a significant amount, resulting in a health risk to patient 12, one or more processors 28 and/or the client application in patient device 24 (and/or another device such as the delivery device itself) may determine (or modify) therapy information prescribing a certain treatment (e.g., insulin) and appropriate amount(s), type(s), and/or schedule(s) to deliver patient 12 in order to sufficiently overcome the expected increase in patient 12's glucose level. Patient 12 or patient 12's caregiver administers the certain treatment according to the above appropriate amount, type, and/or schedule and by doing so, effectively maintains patient 12's glucose level within a healthy range. It should be further noted that, similar to the above determination (and/or modification) of the therapy information, the personalized model may be configured to predict changes in other biomarker levels and such a prediction may be incorporated into patient 12's therapy delivery such that an appropriate amount, type, and/or schedule of a certain treatment may be administered to patient 12 to properly account for the change in the other biomarker levels. It should be noted that the above therapy is specific to patient 12 and may not be effective in other patients.

Some example techniques proactively monitor patient 12's food consumption for any food item that, if consumed, is likely to disrupt patient 12's glucose level where that disruption is likely to negatively affect (e.g., diminish) the effectiveness of the patient 12's diabetes therapy. These example techniques may be beneficial in patient 12 receiving an effective or “correct” dosage of insulin. Even if patient 12 has a treatment schedule for his/her diabetes therapy, patient 12 may eat a food item that increases patient 12's glucose level beyond a point where a next scheduled dosage can reduce back to a healthy level, rendering ineffective the next scheduled dosage. Some example techniques dynamically change one or more parameters of the current treatment schedule, for example, to modify a treatment type, amount, or time in order to be effective against the increased glucose level.

In order to prepare an effective dosage for patient 12's next injection, some example techniques recommend food items and assume patient 12's follows these recommendations. In some examples, one or more processors 28 may simplify a machine learning model to include a set of mappings between particular food items and corresponding food item information and/or therapy information. Some mappings may further indicate, for each particular food item, a likely increase/decrease in patient 12's glucose level that correlates with consuming that particular food item. As an example, food data 27 may include a mapping between a specific volume (e.g., a portion size) of a particular type of meat (e.g., a sirloin steak, chicken breast, chicken thigh, and/or the like) and corresponding nutrition information for that particular food item. While there are a number of different metrics for measuring a food item's nutritional value to patient 12, one or more processors 28 may utilize specific nutritional data attributes (e.g., carbohydrate count) to estimate the likely increase/decrease in patient 12's glucose level. As another example, food data 27 may include a second mapping between the portion size of the particular type of meat and corresponding therapy information. While patient 12 may utilize any one amongst different delivery systems (e.g., a smart injection pen, a syringe, or a pump 14) for diabetes therapy, one or more processors 28 may generate the therapy information to be compatible with patient 12's delivery system. One or more processors 28, in cooperation with patient 12's delivery system, may utilize the therapy information to apply parameters directing patient's therapy, for example, by setting a treatment type, a treatment amount, recommended time(s) for delivering therapy, site(s) on patient 12 for injecting a treatment, and/or the like.

As described herein, patient device 24 and/or cloud 26 may support the above-mentioned determination by maintaining (e.g., in food data 27) food item information for each food item of a plurality of food items. In some examples, food data 27 may be configured to store, for each food item, nutrition information, volume information, and any other food item information to be used in determining how much patient 12's glucose level will likely increase or decrease by consuming that food item. Food data 27 may be configured to distinguish different food items (e.g., by type, such as meat from fruit). Food data 27 may be configured to distinguish specific types of similar food items (e.g., gala apples from green apples, wild-caught salmon from farm-raised salmon, whole milk from skim milk, organic spinach from non-organic spinach, grass-fed beef from corn-fed beef, factory poultry and heritage poultry, and/or the like). To illustrate by way of example, food data 27 may store food item information for different types of apples (e.g., a fuji apple, green apple, gala apple, and/or the like) including a mapping between a volume and an expected carbohydrate count in each type of apple having a similar volume measurement. One or more processors 28 may determine the volume measurement from a mesh model of the single apple, access food data 27 to lookup the above example mapping, and then, use the expected carbohydrate count to determine the likely increase or decrease to patient 12's glucose level. In some examples, food data 27 may specify one or more factors and other indicia of either an increase or a decrease in patient 12's glucose level. Examples of such factors include thresholds, conditions, and other criterion for the food item information to satisfy. For a single particular type of food item, food data 27 may define one or more thresholds/conditions on counts of calories, carbohydrates, grams of fat, and/or other nutritional facts that, if satisfied, indicate an increase or decrease in the glucose level. For example, if a particular type of apple's nutritional content (e.g., carbohydrate count) exceeds respective threshold counts (e.g., daily recommended amounts), consuming that apple is likely to increase patient 12's glucose level. In other examples, food data 27 may define one or more thresholds/conditions on a portion size or a total volume of the food item that, if satisfied, is likely to increase or decrease the glucose level. For example, by providing patient 12 with excessive amounts of calories, carbohydrates, grams of fat, and/or the like, consuming that apple is likely to increase patient 12's glucose level.

In some examples, the various components may determine changes to pre-established therapy delivery based on determination of at least one biomarker level (e.g., glucose level from sensor 20). The present disclosure may describe “therapy” as a reference to patient 12's scheduled diabetes treatments where at certain time(s) the example system delivers an amount of a specific treatment type. Patient 12 may receive this treatment at a fixed time in the day (e.g., noon) or before/during/after an event (e.g., a meal event). As described herein, patient 12's diet may play a significant role in the therapy and depending on which food items are consumed by patient 12, the various components may determine an appropriate treatment type and/or amount to delivery (which may or may not differ from a scheduled treatment type and/or amount).

As described herein, therapy information for patient 12 may include a treatment schedule for insulin pump 14 and/or sensor 20 to execute and examples of such a schedule may indicate planned treatment deliveries of specific amounts of an insulin type. Based on dietary recommendations generated by a trained machine learning model and/or patient 12's diet otherwise, one or more processors 28 may adjust the treatment schedule by modifying a type or an amount of a planned treatment. For example, if patient 12 decides to eat one or more food items with excessive amounts of calories, carbohydrates, grams of fat, and/or the like, causing an unhealthy increase in patient 12's glucose level, one or more processors 28 may modify the therapy information to account for patient 12's food consumption. In some examples, by increasing an amount of insulin, changing a type of insulin, and/or adjusting a time at which insulin is to be delivered in accordance with next planned injection, one or more processors 28 may return patient 12 back to a healthy glucose level. Similarly, if patient 12 decides not to eat (e.g., fast) or restrict his/her diet to food items that are low in calories, carbohydrates, grams of fat, and/or the like, resulting in an unhealthy decrease in patient 12's glucose level, one or more processors 28 may modify the therapy information to account for patient 12's food consumption. In some examples, by reducing an amount of insulin, changing a type of insulin, and/or adjusting a time at which insulin is to be delivered in accordance with next planned injection, one or more processors 28 may return patient 12 back to a healthy glucose level.

For example, patient 12 may forget to cause insulin pump 14 to deliver insulin after eating, resulting an insulin shortfall. Alternatively, patient 12 may cause insulin pump 14 to deliver insulin after eating but may have forgotten that patient 12 previously caused insulin pump 14 to deliver insulin for the same meal event, resulting in an excessive insulin dosage. Also, in examples where CGM 20 is utilized, insulin pump 14 may not take any action until after the glucose level is greater than a target level. By proactively determining that patient 12 is engaging in an activity, insulin pump 14 may be able to deliver insulin in such a manner that the glucose level does not rise above the target level or rises only slightly above the target level (i.e., rises by less than what the glucose level would have risen if insulin were not delivered proactively). In some cases, by proactively determining that patient 12 is engaging in an activity and delivering insulin accordingly, the glucose level of patient 12 may increase more slowly.

In some examples, food data 27 is stored on patient device 24 rather than or in addition to cloud 26. Although FIG. 1 depicts the example system as having wearable device 22 on patient 12's wrist, wearable device 22 may be configured somewhere else on patient 12 (e.g., on patient 12's head), it is possible that other example systems may have fewer, more, and/or different wearable devices 22.

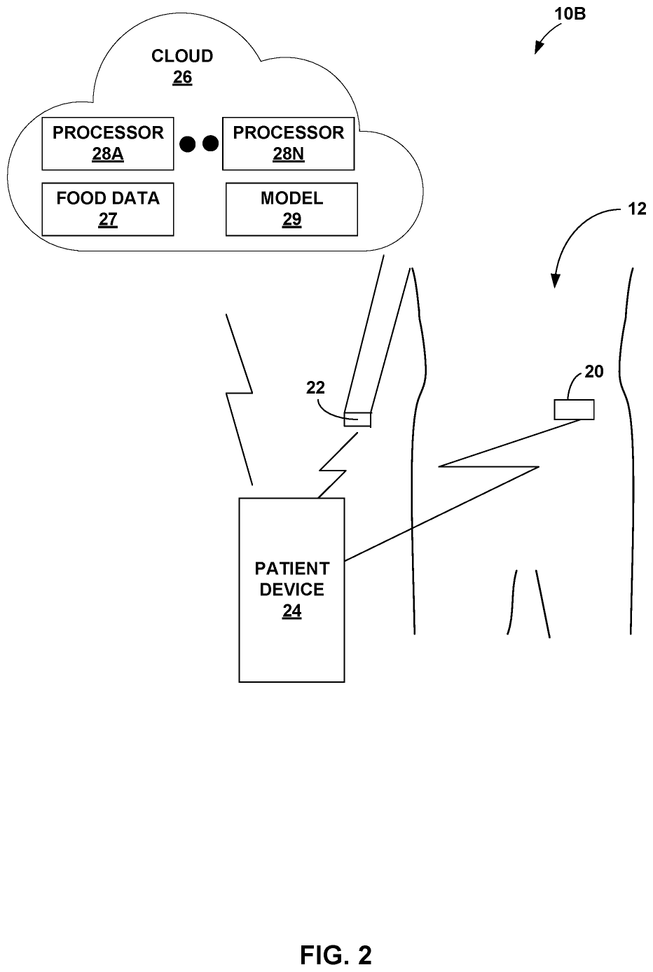

FIG. 2 is a block diagram illustrating another example system for delivering or guiding therapy dosage, in accordance with one or more examples described in this disclosure. FIG. 2 illustrates system 10B that is similar to system 10A of FIG. 1. However, in system 10B, patient 12 may not have insulin pump 14. Rather, patient 12 may utilize a manual injection device (e.g., a syringe) to deliver insulin (not shown). For example, rather than insulin pump 14 automatically delivering insulin, patient 12 (or possible a caretaker of patient 12) may fill a syringe with insulin and inject himself or herself.

Patient 12 utilizes patient device 24 to manage their therapy delivery in a number of ways. Patient device 24 may run various applications that patient 12 may use to ensure that an effective amount of a correct insulin type is loaded into the syringe at a correct time of the day. One example application may be a client application as described herein for a cloud computing service, which may support patient 12 with recommendations with respect an activity (e.g., eating) that patient 12 is undertaking or about to undertake. Patient 12 has access to a plurality of food items and if an inventory of that plurality (or a portion thereof) is known, the client application may apply a trained personalized machine learning model to determine whether each particular food item's nutrient composition is likely to increase or decrease patient 12's biomarker level (e.g., a physiological biometric such as a glucose level, ketone level, and/or the like); and if so, the client application uses the trained personalized machine learning model to estimate by what amount the biomarker level would increase or decrease.

The client application considers each food item to recommend patient 12 based (in part) on the machine learning model's output for that food item. The client application disqualifies from consideration any food item whose consumption is likely to cause a biomarker level of interest to increase or decrease by a significant amount, which may be defined by a pre-determined threshold value. Among the remaining food items under consideration, the client application selects each food item where an estimated biomarker level after consumption satisfies at least one criterion. For example, if the estimated biomarker level of a particular food item is equal to or exceeds a desired biomarker level, the client application selects that particular food item for the recommendation. In another example, the client application selects a food item having lowest estimated biomarker response amongst the remaining food items (e.g., the food item most likely to lower or raise the biomarker level the least). The client application may notify patient 12 of a recommended food item in a number of ways, including generating content for display on a display device of patient device 24. Content may be defined as any visual element for presentation by a computing device and is inclusive of every media form including multimedia (e.g., graphics, text, audio, video, tactile, etc.). Patient device 24 may provide a graphical user interface (GUI) on which the client application may present content (e.g., rich-Internet content (RIC)) in which a combination of GUI elements conveys information describing the recommended food item.

Patient 12 benefits from viewing the content and comprehending the conveyed information, for example, by following that information and consuming the recommended food item (e.g., instead of another and less healthy option). If patient 12 consumes the recommended food item, the biomarker level of interest is expected to be within a healthy range. Patient 12 may use the recommendation to decide between multiple candidates, not necessarily selecting the recommended food item. Patient 12 may rely on the client application to dictate their diet over the near future. The client application may employ a variety of content to provide additional functionality. Patient device 12 may use the client application's recommendations to perform a variety of different tasks. The present disclosure envisions additional examples including commercial uses of the recommendation. As one example, a variety of e-commerce or m-commerce solutions, such as those known to those of ordinary skill, may facilitate food orders on behalf of patient 12 and may do so without human intervention (e.g., autonomously).

To help guide patient 12 in their manual injection device use through his/her diabetes, one or more processors 28 may generate (e.g., for output on an electronic display of patient device 24) content indicative of at least a portion of a recommendation and (modified) therapy information. For example, one or more processors 28 may communicate to patient device 24 an initial treatment schedule of manual injection times for patient 12's reference. Patient device 24 may display the treatment schedule and/or highlight a next scheduled injection of insulin (or another treatment). Patient device 24 may also display a recommended treatment amount and type for each scheduled injection. As described herein, patient device 24 may monitor patient 12's movements (e.g., arm movements) for gestures indicative of a manual injection, particularly at or around a time of the next scheduled injection, in order to determine whether patient 12 took his/her scheduled diabetes treatment. Upon detecting an injection at or around the time of a next scheduled injection, the client application running in patient device 24 and/or one or more processors 28 may confirm consumption of the recommended food item. Confirmation of patient 12's consumption of that food item eliminates doubt when the estimated biomarker level is eventually compared with sensor measurements indicating actual biomarker levels over a time period.

Upon detecting no injection at or around the time of the next scheduled injection, patient device 24 and/or one or more processors 28 may determine that patient 12 forgot and/or missed their next scheduled injection; to alert patient 12 of the possible risk to their health by not following their treatment schedule, patient device 24 may output content warning patient 12 that the next scheduled injection has not been detected.

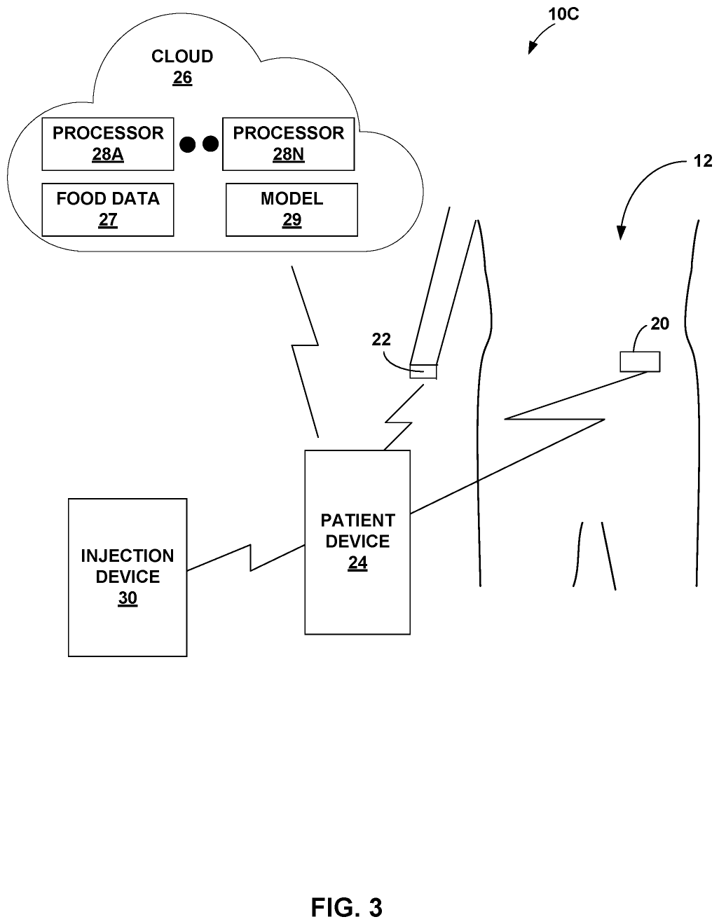

FIG. 3 is a block diagram illustrating another example system for delivering or guiding therapy dosage, in accordance with one or more examples described in this disclosure. FIG. 3 illustrates system 10C that is similar to system 10A of FIG. 1 and system 10B of FIG. 2. In system 10C, patient 12 may not have insulin pump 14. Rather, patient 12 may utilize injection device 30 to deliver insulin. For example, rather than insulin pump 14 automatically delivering insulin, patient 12 (or possible a caretaker of patient 12) may utilize injection device 30 to inject himself or herself.

Injection device 30 may be different than a syringe because injection device 30 may be a device that can communicate with patient device 24 and/or other devices in system 10C. Also, injection device 30 may include a reservoir, and based on information indicative of how much therapy dosage to deliver may be able to dose out that much insulin for delivery. In some examples, injection device 30 may be similar to insulin pump 14, but not worn by patient 12. One example of injection device 30 is an insulin pen (sometimes also called a smart insulin pen) such as the InPen™ smart insulin pen by Medtronic, Inc. Another example of injection device 30 may be an insulin pen with a smart cap attached to it, where the smart cap can be used to determine particular insulin dose amounts and/or delivery.

In some examples, injection device 30 may preset parameters for a scheduled injection to deliver an insulin dose in accordance with therapy information generated by one or more processors of the example system, such as one or more processors 28 of cloud 26. As described herein, cloud 26 may deploy machine learning models (once fully trained) to a client application running in patient device 24 and using injection device 30 to manage their diabetes and/or another affliction.

Compared to system 10A and/or system 10B, one or more processors 28 may follow a similar approach for system 10C when determining which food item(s) to recommend patient 12. As described herein, one or more processors 28 and/or the client application running in patient device 12 may recommend a particular food item based on that particular food item's impact on patient 12, for example, patient 12's nutrition state which may be defined as a set of biomarker levels of interest. In one example, one or more processors 28 and/or the client application running in patient device 12 may recommend a food item having a lowest impact on patient 12's nutrition state. If the food item is a candidate for recommendation, one or more processors 28 and/or the client application running in patient device 12 may use a trained model to determine whether that food item's nutrient composition is likely to cause an increase or decrease in patient 12's biomarker level(s). By comparing multiple food items, one or more processors 28 and/or the client application running in patient device 12 may recommend the food item causing a smallest change in patient 12's biomarker level(s). In another example, one or more processors 28 and/or the client application running in patient device 12 recommend the food item most likely to move patient 12's biomarker level(s) into healthy range(s). In another example, the recommended food item may result in biomarker level(s) expected to be within a desired range.

It should be noted that patient 12's glucose level is one example biomarker level and representative of a portion/component of patient 12's nutrition state at any given point in time; therefore, depending on the particular food item's nutrient composition, consuming that particular food item most likely impacts one or more other biomarkers in patient 12 besides glucose.

One or more processors 28 of cloud 26 may generate therapy information, for example, specifying a time, an amount, and/or a type of treatment delivery with injection device 30. In some examples, cloud 26 leverages resources in food data 27 to identify specific food item information describing each particular food item (e.g., by name (e.g., sirloin steak, ribeye steak, fried chicken breast, grilled chicken breast) and/or by type (e.g., steak, chicken breast, salmon)). In another example, one or more processors 28 of cloud 26 may determine other food item information including nutritional facts (and other nutrition information) and portion sizes (and other volume information) for each particular food item. Cloud 26 may maintain a trained personalized model to determine an expected biomarker level from patient 12 consuming the one or more food items. In some examples, one or more processors 28 may estimate, based on a particular food item's carbohydrate count, an increase in patient 12's glucose level and determine an amount and/or type of diabetes treatment (e.g., insulin) to (most likely) administer, for example, in order to offset the estimated increase.

If patient 12 forgets the scheduled injection, patient device 24 and/or injection device 30 may output an alert notifying patient 12 of the missed dosage and one or more processors 28 may determine whether to modify any of parameters in the therapy information in response to the missed dosage. In some examples, to account for the missed dosage, one or more processors 28 may instruct injection device 30 to adjust a current insulin level available in the reservoir, in preparation of a stronger dose. One or more processors 28 may instruct injection device 30 to change an insulin type. If the scheduled injection was during mealtime, the missed dosage may be appropriate only for mealtime; for at least this reason, injection device 30 may be directed to replace a bolus dosage of insulin with a basal dosage of insulin or vice versa.

The above examples described insulin pump 14, a syringe, and injection device 30 as example ways in which to deliver insulin. In this disclosure, the term “insulin delivery device” may generally refer to a device used to deliver insulin. Examples of insulin delivery device include insulin pump 14, a syringe, and injection device 30. As described, the syringe may be a device used to inject insulin but is not necessarily capable of communicating or dosing a particular amount of insulin. Injection device 30, however, may be a device used to inject insulin that may be capable of communicating with other devices or may be capable of dosing a particular amount of insulin. Injection device 30 may be powered (e.g., battery powered) device, and the syringe may be device that requires no power.

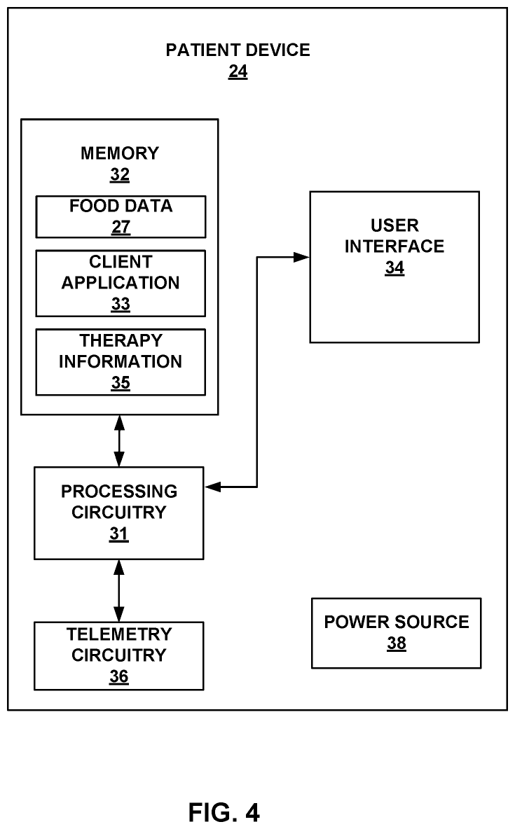

FIG. 4 is a block diagram illustrating an example of a patient device, in accordance with one or more examples described in this disclosure. While patient device 24 may generally be described as a hand-held computing device, patient device 24 may be a notebook computer, a cell phone, or a workstation, for example. In some examples, patient device 24 may be a mobile device, such as a smartphone or a tablet computer. In such examples, patient device 24 may execute an application that allows patient device 24 to perform example techniques described in this disclosure. In some examples, patient device 24 may be specialized controller for communicating with insulin pump 14.

Although the examples are described with one patient device 24, in some examples, patient device 24 may be a combination of different devices (e.g., mobile device and a controller). For instance, the mobile device may provide access to one or more processors 28 of cloud 26 through Wi-Fi or carrier network and the controller may provide access to insulin pump 14. In such examples, the mobile device and the controller may communicate with one another through Bluetooth (BLE). Various combinations of a mobile device and a controller together forming patient device 24 are possible and the example techniques should not be considered limited to any one particular configuration.

As illustrated in FIG. 4, patient device 24 may include a processing circuitry 31, memory 32, user interface 34, telemetry circuitry 36, and power source 38. Memory 32 may store program instructions that, when executed by processing circuitry 31, cause processing circuitry 31 to provide the functionality ascribed to patient device 24 throughout this disclosure.

In some examples, memory 32 of patient device 24 may store food item information including a portion of food data 27 retrieved from cloud 26. As described herein, patient device 24 may store a profile having a set of attributes for each food item, such as a food item type, a carbohydrate count, number of servings, a portion size, a calorie count, etc., as food item information. For each food item that has been eaten or is about to be eaten, example attributes in general may identify that food item (e.g., by name or type) and provide at least one of nutrition information or volume information. Processing circuitry 31 (e.g., via user interface 34) may output various content indicative of at least one of the attributes stored in memory 32. Processing circuitry 31 (e.g., via user interface 34) may receive input specifying any of the above-mentioned attributes and/or other attributes of the plurality of attributes stored in memory 32.

In some examples, client application 33 may include a software program configured to generate a graphical user interface (GUI) and execute logic that provides functionality corresponding to guiding patient 12's therapy, for example, by supporting the proactive monitoring of patient 12's food consumption with access to food data 27 and dynamic adjustment of the patient's 12's treatment type, amount, and/or delivery time. Client application 33 may be mobile application configured to run on a mobile device (e.g., a smartphone) and assist patient 12 in capturing representations of food items and accessing services provided by cloud 26. In some examples, client application 33 on behalf of patient 12 may submit one or more food items and request that one or more processors 28 analyze corresponding food item profiles, recommend at least one for consumption, and (if needed) modify patient 12's current diabetes therapy as codified in therapy information 35.

In some examples, memory 32 of patient device 24 may store in therapy information 35 a plurality of parameters, such as amounts of insulin to deliver, target glucose level, time of delivery etc. Processing circuitry 31 (e.g., through telemetry circuitry 36) may output the parameters stored in memory 32 to insulin pump 14 or injection device 30 for delivery of insulin to patient 12. In some examples, processing circuitry 31 may execute one or more supporting applications in support of patient 12's therapy, such as a notification application that outputs notifications to patient 12, such as notification to take insulin, amount of insulin, and time to take the insulin, via user interface 34.

Client application 33 may communicate similar notifications to a device operated by patient 12's caregiver to which an electronic display may present these notifications. In other examples, the notification application may output content indicative of notifications to patient 12, such as notification to take insulin, amount of insulin, and time to take the insulin, via user interface 36.

In some examples, one or more processors 28 may simplify (e.g., prune) food data 27 to include a set of mappings between particular food items and corresponding food item information and/or therapy information. In some examples, each mapping may provide information for determining when a next insulin dose is to be taken. Some mappings may further indicate, for each particular food item, a likely increase/decrease in patient 12's glucose level that correlates with consuming that particular food item. In some examples, one or more processors 28 may determine a rate that particular food item decreases/increases the patient's glucose level based on information provided by insulin pump 14 and/or sensor 20 and/or information provided by food data 27. For the particular food item or any food item(s) of a specific food type, one or more processors 28 may mathematically combine sensed measurements (e.g., glucose level readings) with corresponding volume information (e.g., discrete portion sizes or a continuous volumetric function) to determine an approximate rate or rates at which, for example, patient 12's glucose level is expected to change. It is well known that foods having a blend of carbohydrates, fats, and proteins will raise blood sugar more slowly than foods that are predominately carbohydrates. To illustrate by way of example, most forms of pizza, regardless of how many slices (e.g., portion sizes), are rich in carbohydrates and fats (e.g., unsaturated fats) and contain considerably above normal amounts of each. Considering each slice to be a discrete portion size, some examples may compute the rate at which patient 12's glucose level changes as a ratio between an expected increase or decrease (e.g., in mg/dL or mmol/L where mmol represents a millimole) and a number of slices. Because of the high fat content in a single pizza slice and especially in a large portion size (e.g., a substantial number of slices or whole pies), patient 12 may digest one or more pizza slices slowly, resulting in a steady and eventually prominent increase in patient 12's glucose level. To overcome or mitigate such an increase, one or more processors 28 may modify patient 12's therapy information 35 to deliver, at a next scheduled treatment, a partial bolus dose of insulin before starting a meal event and then, a remainder of the bolus dose of insulin at least an hour after concluding the meal event. By doing so, one or more processors 28 may improve upon an effectiveness of patient 12's diabetes treatments (e.g., treatment schedule). This may be because patient 12's next treatment (as modified) accounts for the carbohydrates and/or the prolonged digestion process, thereby improving patient 12's diabetes management and health.

In some examples, food data 27 may include a mapping between a specific volume (e.g., a portion size) of meat (e.g., a steak) and corresponding nutrition information for that particular food item. While there are a number of different metrics for measuring a food item's nutritional value to patient 12, one or more processors 28 may utilize specific nutritional data attributes (e.g., carbohydrate count) to estimate the likely changes (e.g., increase or decrease) in patient 12's glucose level. As another example, food data 27 may include a second mapping between the portion size of the steak and corresponding therapy information. While patient 12 may utilize any one amongst different delivery systems (e.g., a smart injection pen, a syringe, or a pump 14) for diabetes therapy, one or more processors 28 may generate the therapy information to be compatible with patient 12's delivery system. One or more processors 28, in cooperation with patient 12's delivery system, may utilize the therapy information to apply parameters directing patient's therapy, for example, by setting a treatment type, a treatment amount, recommended time(s) for delivering therapy, site(s) on patient 12 for injecting a treatment, and/or the like.

Memory 32 may include any volatile, non-volatile, fixed, removable, magnetic, optical, or electrical media, such as RAM, ROM, hard disk, removable magnetic disk, memory cards or sticks, NVRAM, EEPROM, flash memory, and the like. Processing circuitry 30 can take the form one or more microprocessors, DSPs, ASICs, FPGAs, programmable logic circuitry, or the like, and the functions attributed to processing circuitry 30 herein may be embodied as hardware, firmware, software or any combination thereof.

User interface 34 may include a button or keypad, lights, a speaker for voice commands, and a display, such as a liquid crystal (LCD). In some examples the display may be a touch screen. As discussed in this disclosure, processing circuitry 30 may present and receive information relating to therapy via user interface 34. For example, processing circuitry 30 may receive patient input via user interface 34. The patient input may be entered, for example, by pressing a button on a keypad, entering text, or selecting an icon from a touch screen. The patient input may be information indicative of food that patient 12 eats, such as for the initial learning phase, whether patient 12 took the insulin (e.g., through the syringe or injection device 30), and other such information.

Telemetry circuitry 36 includes any suitable hardware, firmware, software or any combination thereof for communicating with another device, such as cloud 26, insulin pump 16 or injection device 30, as appliable, wearable device 22, and CGM 20. Telemetry circuitry 36 may receive communication with the aid of an antenna, which may be internal and/or external to patient device 24. Telemetry circuitry 36 may be configured to communicate with another computing device via wireless communication techniques, or direct communication through a wired connection. Examples of local wireless communication techniques that may be employed to facilitate communication between patient device 24 and another computing device include RF communication according to IEEE 802.11 or Bluetooth specification sets, infrared communication, e.g., according to an IrDA standard, or other standard or proprietary telemetry protocols. Telemetry circuitry 36 may also provide connection with carrier network for access to cloud 26. In this manner, other devices may be capable of communicating with patient device 24.

Power source 38 delivers operating power to the components of patient device 24. In some examples, power source 38 may include a battery, such as a rechargeable or non-rechargeable battery. A non-rechargeable battery may be selected to last for several years, while a rechargeable battery may be inductively charged from an external device, e.g., on a daily or weekly basis. Recharging of a rechargeable battery may be accomplished by using an alternating current (AC) outlet or through proximal inductive interaction between an external charger and an inductive charging coil within patient device 24.

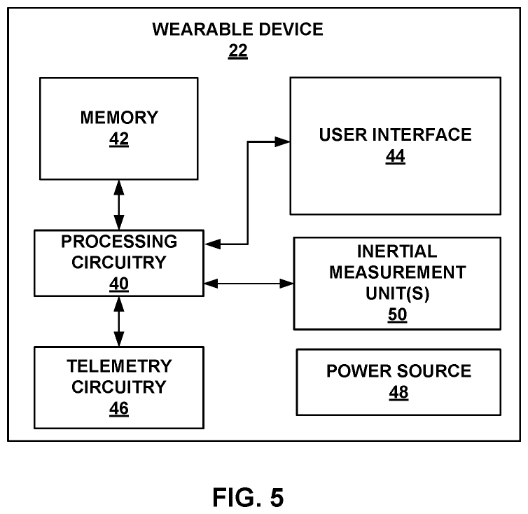

FIG. 5 is a block diagram illustrating an example of a wearable device, in accordance with one or more examples described in this disclosure. As illustrated, wearable device 22 includes processing circuitry 40, memory 42, user interface 44, telemetry circuitry 46, power source 48, and one or more inertial measurement units. Processing circuitry 40, memory 42, user interface 44, telemetry circuitry 46, and power source 48 may be similar to processing circuitry 30, memory 32, user interface 34, telemetry circuitry 36, and power source 38 of FIG. 3, respectively.

Accelerometers and gyroscopes are examples of inertial measurement units 50 and may include various components to determine a pitch-roll-yaw, and x-y-z coordinates of wearable device 22. In some examples, inertial measurement units 50 may be considered as a six-axis inertial measurement unit. For example, inertial measurement units may couple a 3-axis accelerometer with a 3-axis gyroscope. The accelerometer may measure linear acceleration, while the gyroscope may measure rotational motion. Inertial measurement units 50 may output such information (e.g., pitch-roll-yaw and x-y-z coordinates) of the arm of patient 12 to processing circuitry 40. Telemetry circuitry 46 may then output the information from processing circuitry 40 to patient device 24. Patient device 24 may forward the information to one or more processors 28 that can use the information to determine if patient 12 is eating (e.g., if a meal event is occurring).

Processing circuitry 40 may be configured to determine one or more movement characteristics based on values from inertial measurement units. For example, processing circuitry 40 may determine based on values from inertial measurement units if patient 12 is moving his or her arm upwards, downwards, leftwards, rightwards, forwards, backwards, or some combination, including values related to frequency, amplitude, trajectory, position, velocity, acceleration, and/or pattern of movement. Processing circuitry 40 may determine based on values from inertial measurement units 50 orientation of the arm of patient 12, such as whether the back of the hand or the front of the hand is facing patient 12, or if a side of the hand is facing patient 12, such that the thumb is facing patient 12 and the side of the index finger is visible.

As one example, when patient 12 is holding chopsticks to eat, patient 12 may orient his or her wrist in a particular manner, which may be different than if patient 12 is holding a sandwich. The frequency of patient 12 moving his or her arm from a position where he or she is reaching food to a position where he or she is placing food in mouth may be different for different types of food. For example, the frequency and pattern of movement of eating with a fork may be different than eating with a spoon or a knife and fork, which may be different than eating with hands, like a sandwich or pizza. For all of these different food items, there may be a difference in the movement characteristics, and different output values from inertial measurement units. However, for all of the movement characteristics, one or more processors (including processing circuitry 40 in some examples) may be configured to determine that patient 12 is eating. In some examples, the one or more processors may use contextual information (e.g., time of day, location, detection of a pre-meal bolus) to determine that patient 12 is eating, as described above.

Wearable device 22 represents any wearable device that is described herein or conceivable in view of the present disclosure. FIGS. 1-3 depict wearable device 22 as a smart watch but wearable device 22 is not so limited. Another example of wearable device 22 may be smart glasses.

Wearable device 22 may include any available (image) sensing technology such as a camera capable of providing two-dimensional data and/or a LiDAR sensor capable of providing three-dimensional data in any format including point clouds—which may alternatively be referred to as “point cloud data” or “point cloud data sets” in the present disclosure. The LiDAR sensor may produce LiDAR sensor data including point cloud data in the format of (x, y, z, r) where x, y, and z are 3D coordinates and r is reflectivity (the intensity of the reflected laser light). LiDAR sensor may generate point clouds to represent a region in three-dimensional space where each three-dimensional data cube (e.g., voxel) maps to a two-dimensional area in a camera image of the above-mentioned image data.

One or more processors 28 of cloud 26 may leverage wearable device 22 to generate image data for an object that is a suspected food item. By comparing the object's image data to pre-determined image data in food data 27, one or more processors 28 may identify which food item is being represented by the object. In some examples, one or more processors 28 may identify a food type for the object in addition or as an alternative to identifying the particular food item represented by the object. The food item's image data may be used for determining one or more nutritional facts.

FIG. 6A and FIG. 6B are illustrations of a packaging structure for kit 60. As described herein, a diabetes patient such as patient 12 may use kit 60 to generate a trained personalized machine learning model. Kit 60 may include or operate with a therapy delivery device, such as those depicted in FIGS. 1-3.

As depicted in FIG. 6A, kit 60 include sensing box 61 with on-boarding materials and a set of baseline food items herein referred to as baseline bars 62 or, simply, bars 62. Sensing box 61 may function as a housing for a biomarker sensor (including a sensor inserter). As described herein, the biomarker sensor is configured to measure current levels of a biomarker of interest. Sensor 20, which is illustrated in FIGS. 1-3, is one example of a glucose sensor. Other types of devices may be instrumented as biomarker sensors. FIG. 6A depicts baseline bars 62 as examples of the baseline food items described herein. It should be noted that alternative examples of kit 60 may include beverages or a mixture of bars and beverages as examples of the baseline food items described herein.

As described herein, for example, regarding the baseline food items in food data 27 of cloud 26 of FIG. 1, bars 62 represent an inventory of pre-determined baseline food items for a patient's consumption; each bar 62 facilitate a machine learning model's training/calibration. An example model undergoing the above training/calibration benefits (e.g., with respect to accuracy) from having known nutritional facts for each bar 62. Once fully trained/calibrated, the trained/calibrated model may be used to accurately predict patient 12's biomarker level(s) of interest. Similar to food data 27 which maintained a pre-defined profile with known nutritional facts as attributes for one example baseline food item, bars 62 correspond to attributes defining each bar by type using a data set (e.g., a tuple) of known or pre-determined nutritional facts. By “nutritional facts”, the present disclosure refers to each bar 62's nutrient composition; among the considerable examples of “nutritional facts” that could potentially be used for bars 62, counts of calories, carbohydrates, proteins, and other nutrients are among some examples.

In some examples, when kit 60 includes more a plurality of bar 62, kit 60 may also include instructions in printed form and/or via a computerized document regarding how to consume bars 62. Patient 12 may be instructed to consume bars 62 in accordance with a schedule of mealtimes and one or more time intervals between mealtimes. At each mealtime, patient 12 is directed to consume individual bars 62 or pairs of bars 62, for example, in a specific ordering. Patient 12 may also be instructed to avoid certain foods and consume other foods during mealtimes or in-between mealtimes.

An individual bar 62 of a certain type may be similar (e.g., in mass and/or volume) to other bars 62 of the same bar type and therefore, each has a standardized set of nutrition attributes. In some examples, bars 62 may be partitioned into meals (e.g., breakfast, lunch, snack, and dinner) and each meal's corresponding bars 62 may be identical or nearly-identical (e.g., of an equivalent food type and/or have known ingredients. By neutralizing most variables and focusing on patient 12's physiology, a reduction in typical training/calibration time is achieved. This is because other models must account for typical food and meal differences when modeling biomarker response(s). When it is known that patient 12's biomarker response substantially results from consuming an example bar 62 and deriving food energy through decomposition of example bar 62's nutrients, patient 12 most likely will have a similar or proportional response each time that particular bar 62's nutrients are consumed and that relationship can be leveraged in a manner that benefits patient 12, for example, by enhancing patient 12's insulin treatment's efficacy. Because the above model calibration/training may be focused on patient 12's physiological state, a trained model configured to predict patient's biomarker responses may be considered a personalized model.

On patient 12's behalf, a clinician may avail the trained model to make more effective patient 12's diabetes therapy in general, for example, by way of a device for determining (but not administering) a precise bolus or basal dosage to support patient 12's physiological mechanisms in metabolizing example bar 62's nutrients. The device may be an insulin delivery device or may communicate the precise bolus or basal dosage amounts to patient 12's insulin delivery device (e.g., an insulin pump). Patient 12 may benefit from having such information programmed into their personal insulin delivery device, for instance, from avoiding common problems associated with inaccurate insulin treatments.

FIG. 6B depicts additional components of kit 60 including welcome pack 63, sensor box insert 64, baseline bars insert 65, and discovery box 66. Welcome pack 63 may include a welcome booklet, stickers, a sensor user guide, and a disposable bag. Baseline bars insert 64 secures baseline bars 62 in kit 60. As described herein, baseline bars 62 may be used in training the personalized machine learning model. When patient 12 consumes one or more of baseline bars 62, one or more biomarker sensors may record sensor data including one or more biomarker levels. One or more processors and/or a client application may apply an untrained model to the consumed baseline bar's nutritional facts, estimate one or more biomarker levels that are likely to result in response to the patent's consumption, and compare the one or more estimated biomarker levels to the recorded sensor data. Based on that comparison, the one or more processors and/or the client application may adjust the untrained model to improve upon an accuracy of the one or more biomarkers.

Baseline bars insert 67 may be designed to fit a number of baseline bars 62 provided with kit 60. Baseline bars insert 67 is depicted as including three bars, which may be a portion of baseline bars 62 or an entirety of baseline bars 62. In some examples, the personalized model may be considered fully trained when the patient consumes the last one of baseline bars 62. In other examples, training may proceed regardless of the number of baseline bars 62. As illustrated, sensor box insert 65 is to secure sensor box 61 in discovery box 66. Sensor box 61 may be reusable. Discovery box 66 may be configured with a tray magnetic flap closure and also may be reusable.

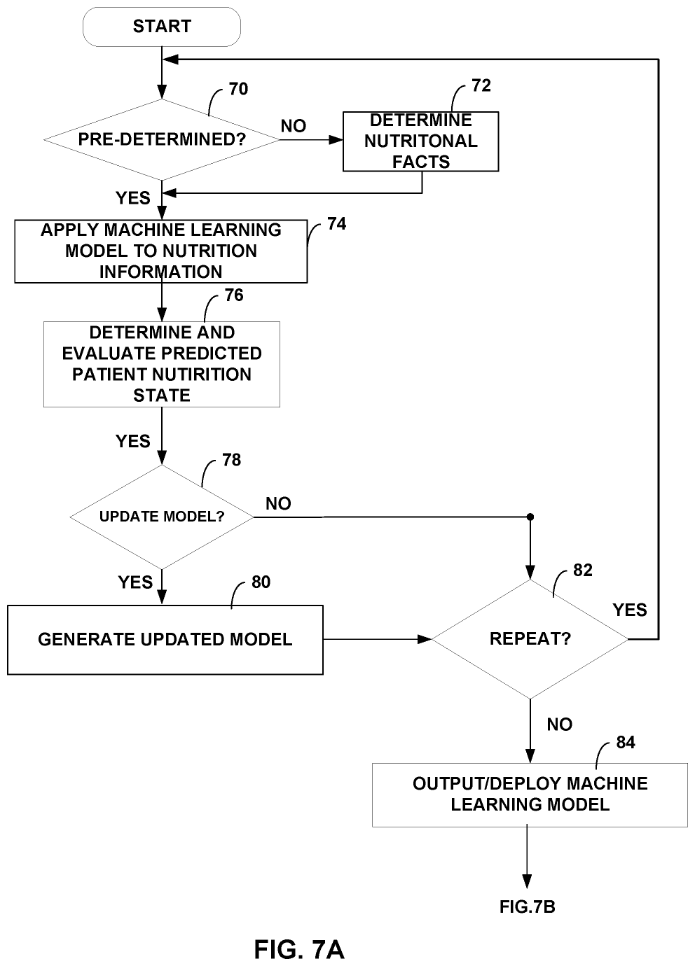

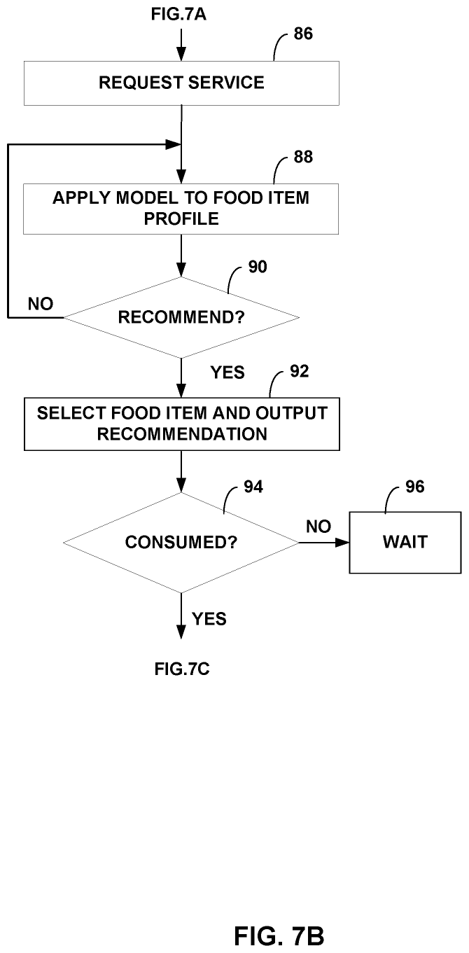

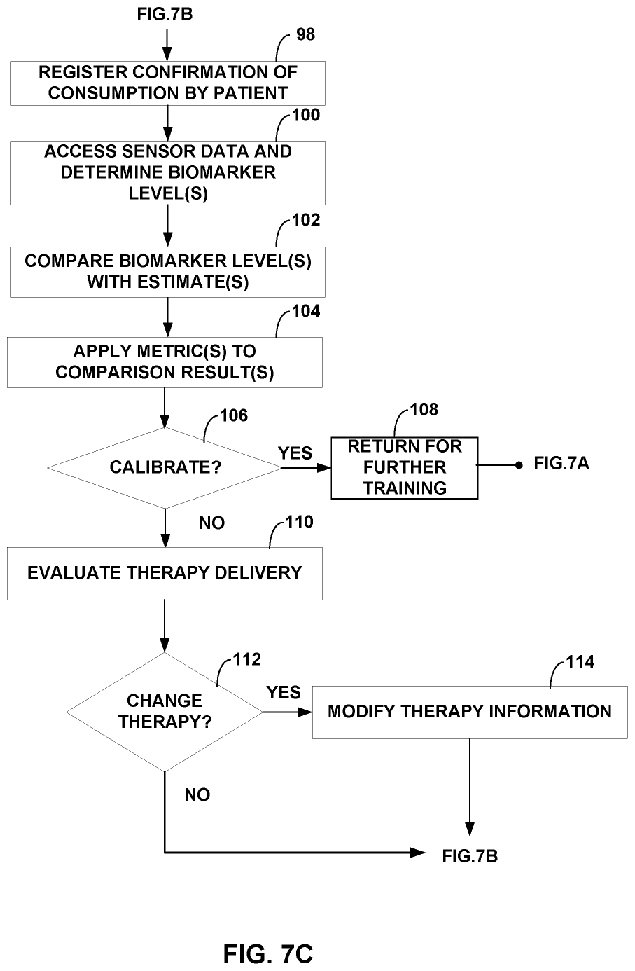

FIGS. 7A-7C are each flow diagrams and in sequence, illustrate an example method for delivering or guiding therapy dosage, in accordance with one or more examples described in this disclosure. As described herein, a patient (e.g., patient 12) may benefit from a device (e.g., wearable device 22, patient device 24, and/or cloud 26) performing the example method, for example, by proactive monitoring of the patient's food consumption and (if needed) adapting the patient's diabetes therapy delivery.

FIG. 7A is a flow diagram illustrating an example training technique for a machine learning model, in accordance with one or more examples described in this disclosure. Example training technique may be performed on a model to personalize that model for a patient (e.g., patient 12) using his/her device(s), which includes one or more of wearable device 22, patient device 24, and/or cloud 26 as described in FIGS. 1-5. Example training technique may be performed with kit 60 as described with respect to FIGS. 6A-6B. FIGS. 7A-7C is described with respect to one or more processors. The one or more processors may be one or more processors 28 of cloud 26, one or more processors of patient device 24 (e.g., processing circuitry 31), one or more processors of wearable device 22 (e.g., processing circuitry 40), one or more processors of insulin pump 14 (if available), one or more processors of injection device 30 (if available), or any combination thereof.

As described herein, the patient (e.g., patient 12) may benefit from wearable device 22, patient device 24, and/or cloud 26 performing the example training technique, for example, by having a trained personalized machine learning model. Such a model may estimate a biomarker level that is tailored to the patient's physiology based on the patient's current biomarker level and the patient consuming a particular food item. For at least that reason, the personalized model (once trained) achieves a higher level of accuracy than generic models and can help the patient better control his or her nutritional state. While the patient may have access to any combination of devices, FIG. 7 is described with respect to devices similar to wearable device 22, patient device 24, and/or cloud 26.

Cloud 26 may support the example training technique, for example, by providing the patient's devices with access to food data 27, computing services provided by one or more processors 28, and machine learning models stored in model 29. Client application 33 of patient device 24 supports the example training technique, for example, by controlling the patient's diet, for example, in cooperation with cloud 26. Cloud 26 may further support the example technique, for example, by providing a service that is configured to accurately predict a particular food item's impact on the patient's biomarker level(s) (e.g., glucose level) based on the particular food item's nutritional content and/or portion size or volume. If a food item's identity (e.g., name or type) is not known or provided (e.g., by the patient), the example training technique may leverage services enabling accurate identification of any food item that the patient is eating or is about to eat. Some services leverage machine learning techniques that use mesh models of different food items for training a machine learning model to accurately identify one or more food items in a mesh model that may or may purport to be any particular food item.

In general, the example training technique may commence at point such as when the one or more processors are provided with an inventory of food items to be used in training the model. Nutrition information for these food items, which may be referred to as baseline food items, may have known and/or pre-determined before commencing the training technique as described above.

The patient may initiate the example training technique by consuming a food item, which may or may not be a baseline food item as described herein. The above inventory may identify which food items have pre-determined and/or known nutritional information and which food items do not. Based on a determination that the food item has pre-determined and/or known nutritional information for use in the example training technique (70), that food item may be considered a baseline food item. Based on a determination that the food item does not have predetermined nutritional information about the food item (NO of 70), the one or more processors may proceed to determine one or more nutritional facts about the food item (72). As described herein, nutritional facts may be used as input features for the above machine learning model. There are a number of mechanisms available that may be employed to determine for example a calorie count, carbohydrate amount, and/or any other macronutrient or micronutrient information for the particular food item.

When sufficient nutrition information is available (YES of 70), the one or more processors may proceed to apply the machine learning model to the nutrition information (74). The one or more processors may determine and evaluate a predicted patient nutrition state in response to the patient consuming the food item (76). Based on the predicted patient nutrition state, the one or more processors may determine whether to update the model as part of the example training technique (78). Based on a determination to update the model (YES of 78), one or more processors may generate an update model (80), for example, by adjusting model data to improve upon the accuracy of the model's predictions. Based on a determination not to update the model (NO of 78), the one or more processors may proceed to a determination as to whether to repeat the training technique on another food item in the inventory (82). Based on a determination to resume training the model and repeat the example training technique on the next food item (YES of 82), the one or more processors returns to the determination as to whether the next food item is a baseline food item and waiting for the patient to consume the next food item. Based on a determination not to repeat the example training technique, for example, because the model is sufficiently trained (NO of 82), the one or more processors outputs the trained model (84). In some examples, the one or more processors may deploy the trained model to the patient device for use by the client application in recommending healthy food items for the patient to consume.

FIG. 7B is a flow diagram illustrating an example evaluation technique for a machine learning model, in accordance with one or more examples described in this disclosure.

Similar to FIG. 7A, FIG. 7B is described with respect to one or more processors and a patient. The one or more processors may be one or more processors 28, one or more processors of patient device 24 (e.g., processing circuitry 32), one or more processors of wearable device 22 (e.g., processing circuitry 40), one or more processors of insulin pump 14 (if available), or any combination thereof. The patient may be a diabetes patient similar to patient 12 and the patient's device(s) include one or more of wearable device 22, patient device 24, and/or cloud 26 and possibly one or more additional devices. The patient may be a client of cloud 26 and the patient device may run client application 33. In some examples, the one or more processors may deploy the trained model to a server or cloud that is accessible by the client application for recommending healthy food items for the patient to consume.

While FIG. 7B is depicted as following FIG. 7A, in some examples, the example evaluation technique may employ a trained machine learning model that is not personalized for the patient's physiology.

As illustrated in FIG. 7B, the one or more processors may commence the example evaluation technique by requesting a computing service from cloud 26 to recommend a food item for consumption by the patient (86). In some examples, the request may contain information about one or more food items (e.g., one or more food items the user is considering consuming). The one or more processors may request that the cloud service apply the trained machine learning model to input features in a food item profile for each of one or more food items under consideration for recommendation (88). In cloud 26, one or more processors 28 may directly apply the trained machine learning model to food data 27 and evaluate the model's output data according to one example. In some examples, cloud 26 may deploy (fully trained) personalized models to the patient's device (e.g., patient device 24) and once deployed, a client application in the device may apply the trained personalized machine learning model to a food item profile storing information to be used as input features. Application of the above models may produce output data describing the patient's predicted nutrition state. The patient's nutrition state may be defined as a biomarker level or a combination of multiple biomarker levels and the trained machine learning model may be configured to output estimated levels for each biomarker. As described herein, the patient's predicted nutrition state may further include a likelihood that a food item's nutrient composition causes an increase or decrease in the patient's current biomarker level(s) after the patient consumes the food item. If the patient is eating a banana as one example, the trained machine learning model may be configured to output information indicating what the patient's nutrition state is likely to be if the patient finishes consuming the banana. In this manner, the trained machine learning model determines the patient's likely physiological response to consuming the food item.

Given the number of candidate food items for determining a recommendation, the one or more processors may use corresponding food item profiles and the trained machine learning model to determine if any of these food items should be recommended for consumption by the patient (90). The one or more processors may do so by applying the trained model to each food item profile, evaluating the model's estimated changes to the current biomarker level(s) in response to consuming each food item, and determining whether each food item is safe and/or healthy for the patient's consumption. These estimated changes to the patient's current biomarker level(s) are predicted to occur if the patient actually consumes a corresponding food item within a pre-determined and/or configurable time period. The one or more processors may compare the model's predictions to each other and based on that comparison, identify at least one food item to recommend (e.g., the recommendation may include more than one food item for the patient to choose from). As described herein, a food item profile describes a food item in terms of nutrition information (e.g., nutritional facts) and may distinguish that food item amongst the number of food items that are candidates for recommendation.

Based on the above output data produced by the machine learning model, the one or more processors determine not to recommend any particular food item (NO of 90). In some examples, the one or more processors may request that the computing service find another food item to evaluate for a possible recommendation (e.g., a food item that may not have been included in the request). The computing service may access an inventory of food items and select a new (e.g., next) food item to evaluate. In some examples, the computing service may access an inventory of similar category of foods (e.g., evaluate different types of meats (e.g., sirloin steak, ribeye steak, chicken leg, chicken breast, lamb), vegetables, fruits, grains, and/or the same food category/type as in the request but prepared differently (e.g., grilled chicken breast instead of fried chicken breast)).

Based on determining that a particular food item is to be recommended for consumption (YES of 90), the one or more processors may proceed to select that food item and output a recommendation describing that particular food item (92). In some examples, the one or more processors may proceed to select two or more food items and output a recommendation describing those selected food items. The one or more processors may identify the particular food item(s) to recommend and output various GUI content for presentation to the patient in a manner that the patient is notified according to one example. The one or more processors may select that particular food item based on a corresponding predicted nutrition state that is generated by the trained machine learning model in case the patient actually consumes the food item.

The one or more processors may determine whether the patient actually consumed a recommended food item (94). As described herein, there are a number of mechanisms whose functionality is operative to detect/identify an activity that the patient is undertaking. The one or more processors may employ one or more of these mechanisms to monitor the patient (e.g., patient movement). One of these mechanisms may determine that the patient is eating the particular food item, detect when the patient eventually finished consuming that food item, and then, notify the one or more processors in some manner. The one or more processors may register such a notification as a confirmation of actual consumption by the patient. In this manner, the example method may cause the patient's diabetes therapy to be modified without considering a possibility that the patient did not actually consume the food item and therefore, any estimate of the patient's biomarker level will be inaccurate.

Based on the determination that the patient has not yet consumed the recommended food item (NO of 94), the one or more processors may wait for confirmation from the patient (96). If the one or more processors do not register such a confirmation within a certain amount of time, the one or more processors may wait for one of the above mechanisms to detect the patient's consumption and send a notification message. Based on registering the confirmation that the patient consumed the recommended food item (YES of 94), the one or more processors proceed to evaluate and possibly change the patient's therapy delivery to be more effective in view of the patient's consumption of the recommended food item. In one example, the one or more processors may determine and/or modify therapy information in view of the patient's physiological response to the recommended food item's nutritional composition.

FIG. 7C is a flow diagram illustrating an example operation for guiding a patient's therapy, in accordance with one or more examples described in this disclosure. The example operation may be a continuation of an evaluation technique, such as the example evaluation technique of FIG. 7B.

Similar to FIG. 7A and FIG. 7B, FIG. 7C is described with respect to one or more processors and a patient. The one or more processors may be one or more processors 28, one or more processors of patient device 24 (e.g., processing circuitry 32), one or more processors of wearable device 22 (e.g., processing circuitry 40), one or more processors of insulin pump 14 (if available), or any combination thereof. The patient may be a diabetes patient similar to patient 12 and the patient's device(s) include one or more of wearable device 22, patient device 24, and/or cloud 26 and possibly one or more additional devices. The patient may be a client of cloud 26 and the patient device may run client application 33.

The example operation may commence by first determining whether the trained machine learning model is sufficiently accurate or needs calibration. The model should be sufficiently trained to not only predict the patient's nutrition state but also influence the patient's therapy delivery. If not, the model may undergo further training. After the one or more processors register a confirmation of consumption by the patient (98), the one or more processors may access sensor data and determine actual biomarker levels (100). The one or more processors may compare the actual biomarker levels with estimated biomarker levels determined by the above machine learning model (102).

The one or more processors may apply metrics to comparison results and determine the model's level of accuracy (104). The one or more processors may then determine whether the machine learning model is to be calibrated (106). One example metric may be configured to analyze the differences between the actual biomarker level after consuming a particular food item and the estimated biomarker levels produced by the machine learning model in order to properly assess the model's level of accuracy. Depending on the model's level of accuracy, the one or more processors may determine that the model's level of accuracy is inadequate and therefore, the model is in need of calibration (YES of 106) and return the model for further training (108). As illustrated, the one or more processors may return to the example training technique depicted in FIG. 7A.

On the other hand, the one or more processors may determine that the model's level of accuracy is adequate and not in need of calibration (NO of 106). The one or more processors may proceed to evaluate the patient's therapy delivery (110). As described herein, the patient and the patient device may utilize certain therapy information to direct the patient's therapy delivery. In one example, the one or more processors may assess the therapy information for its effectiveness in the patient's treatment (e.g., diabetes management).

The one or more processors may determine whether to change the patient's therapy by modifying the therapy information at the patient device (112). The one or more processors may change the patient's therapy in view of the patient's recent consumption of the recommended food item (YES of 112). This is because the patient's next treatment should be effective in maintaining healthy biomarker levels against the predicted increase/decrease in the patient's biomarker level(s). The one or more processors may determine that the patient's current therapy prescribes an ineffective treatment in response to the patient's current diet and proceeds to modify the above therapy information (114). The one or more processors may modify the next treatment to be more effective in maintaining healthy biomarker levels.

On the other hand, the one or more processors may determine that the patient's current therapy, as outlined in the therapy information, is proper for the patient's diabetes management and for at least that reason, is not to be changed (NO of 112). Despite the patient's recent food item consumption, the patient's biomarker levels are expected to be within a healthy range and for at least that reason, the one or more processors do not change the patient's current therapy. The one or more processors may proceed to evaluating more food items to possibly recommend the patient for consumption. As illustrated, the one or more processors may return to the example evaluation technique depicted in FIG. 7B.

To illustrated by way of example, each food item consumed by the patient contributes to an overall carbohydrate count for the patient. Upon determining each food item's contribution of carbohydrates (if any) and appropriately modifying that patient's diabetes therapy, for example, by titrating a next dosage of insulin based on that carbohydrate determination. If the patient has a manual injection device (e.g., a syringe), the one or more processors may output data indicating the next dosage amount and/or schedule to the patient (e.g., via a mobile device such as patient device 24). If the patient has an insulin smart pen device, an insulin pen with a smart cap, or another example of injection device 30, the one or more processors may output data indicating the next dosage to injection device 30 and, in turn, injection device 30 may automatically prepare the next dosage (if possible). If the patient has an insulin delivery device such as an example of insulin pump 14, the one or more processors may output data indicating the next dosage amount and/or schedule to the insulin delivery device, which may or may not automatically administer to the patient.

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including one or more processors 28 of cloud 26, one or more processors of patient device 24, one or more processors of wearable device 22, one or more processors of insulin pump 14, or some combination thereof. The one or more processors may be one or more integrated circuits (ICs), and/or discrete electrical circuitry, residing in various locations in the example systems described in this disclosure.

The one or more processors or processing circuitry utilized for example techniques described in this disclosure may be implemented as fixed-function circuits, programmable circuits, or a combination thereof. Fixed-function circuits refer to circuits that provide particular functionality, and are preset on the operations that can be performed. Programmable circuits refer to circuits that can be programmed to perform various tasks, and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. The processors or processing circuitry may include arithmetic logic units (ALUs), elementary function units (EFUs), digital circuits, analog circuits, and/or programmable cores, formed from programmable circuits. In examples where the operations of the processors or processing circuitry are performed using software executed by the programmable circuits, memory accessible by the processors or processing circuitry may store the object code of the software that the processors or processing circuitry receive and execute.

Various aspects of the disclosure have been described. These and other aspects are within the scope of the following claims.

What is claimed is:

1. A system comprising:
memory configured to store a model; and
processing circuitry communicatively coupled to the memory, wherein the processing circuitry is configured to:
execute a training process for the model to predict a patient nutrition state of a patient based on a predetermined food item consumed by the patient within a time period, wherein to execute the training process, the processing circuitry is further configured to:
determine an estimated biomarker level based on the predetermined food item profile having a set of nutritional attributes for the food item and the model;
receive an actual biomarker level of the patient after the patient consumes the food item within the time period; and
calibrate the model based on comparing the estimated biomarker level to the actual biomarker level;
repeat the training process for one or more food items of a set of predetermined food items; and
output the trained model.

2. The system of claim 1, wherein to calibrate the model, wherein the processing circuitry is further configured to register a confirmation that the patient consumed the predetermined food item within the time period.

3. The system of claim 1 further comprising processing circuitry configured to apply the model or the trained model to a food item having a known food type and unknown nutrition attributes.

4. The system of claim 1, wherein to repeat the training process for the one or more food items, the processing circuitry is further configured to repeat the training process until an accuracy metric for the model is within a threshold value.

5. The system of claim 1 further comprising processing circuitry configured to output, for displaying, instructions for the patient, the instructions comprising a schedule for consuming each of the set of food items.

6. The system of claim 1 further comprising processing circuitry configured to:
determine a first predicted patient nutrition state of the patient based in part on applying the trained model to a first food item profile corresponding to a first food item, wherein the corresponding food item profile comprises a set of nutritional attributes for the selected food item and wherein the trained model determines, for the set of nutritional attributes, at least one estimated biomarker level expected to be within at least one desired range if the patient consumes the first food item;
select the first food item to recommend for the patient based on the first predicted patient nutrition state; and
generate, for display, output data indicating the selected food item.

7. The system of claim 1 further comprising processing circuitry configured to:
apply the model to each food item profile of a second set of food item profiles to determine a predicted patient nutrition state for each food item of a second set of food items, wherein each predicted patient nutrition state of the patient comprises at least one estimated biomarker level based on a delivery device directing the therapy delivery and the patient consuming a particular food item within the time period; and
identify, from amongst the second set of food items, a food item to recommend to the patient based on a comparison between a corresponding predicted patient nutrition state and a desired patient nutrition state of the patient.

8. A system comprising:
memory configured to store a trained model; and
processing circuitry communicatively coupled to the delivery device, wherein the processing circuitry is configured to:
apply the trained model to one or more food item profiles to generate a predicted patient nutrition state for each of the one or more food item profiles, wherein the trained model generates each predicted patient nutrition state of a patient based on a set of nutritional attributes for a corresponding food item;
select, without patient input, one or more food items associated with the one or more food item profiles to recommend for consumption by the patient based at least on predicted patient nutrition states of the patient corresponding to the one or more food item profiles, wherein the predicted patient nutrition state comprises at least one estimated biomarker level expected to be within at least one desired range if the patient consumes the selected food item; and
generate, for display, output data indicating the selected one or more food items.

9. The system of claim 8, wherein to select the food item, the processing circuitry is further configured to:
identify, from amongst the set of food items, the selected one or more food items to recommend based on a comparison between the predicted patient nutrition state and a desired patient nutrition state.

10. The system of claim 8 further comprising processing circuitry configured to:
train the trained model by, for a baseline food item of a set of predetermined baseline food items, applying a model to a corresponding baseline food item profile having a predetermined set of nutritional attributes for that baseline food item, wherein the processing circuitry is further configured to train the model for each baseline food item in the set of predetermined baseline food items or until an accuracy metric is within a threshold value.

11. The system of claim 10 further comprising processing circuitry configured to:
determine an estimated biomarker level based on the patient consuming a particular baseline food item of the set of predetermined baseline food items; and
calibrate the model based on comparing the estimated biomarker level to an actual biomarker level after the patient consumes the particular baseline food item within a time period.

12. The system of claim 8 further comprising:
a delivery device configured to execute therapy information for directing therapy delivery for the patient; and
wherein the processing circuitry is configured to select, without the patient input, the one or more food items to recommend for the consumption by the patient based on one or more predicted patient nutrition states of the patient corresponding to one or more food item profiles, wherein the predicted patient nutrition state comprises at least one estimated biomarker level expected to be within at least one desired range if the delivery device executes the therapy information and the patient consumes the selected food item.

13. A method performed by a medical system having memory for storing a trained model, the method comprising:
applying, by processing circuitry of the medical system, a trained model to one or more food item profiles to generate a predicted patient nutrition state of a patient for each of the one or more food item profiles;
selecting, by the processing circuitry, one or more food items to recommend for consumption by the patient based at least on the predicted patient nutrition state after the patient consumes each of the one or more selected food items, wherein the predicted patient nutrition state comprises at least one estimated biomarker level expected to be within at least one desired range if the patient consumes the one or more selected food items; and
generating, by the processing circuitry, output data for display indicating the selected one or more food items.

14. The method of claim 14, wherein selecting, by the processing circuitry, the one or more food items to recommend further comprises selecting, by the processing circuitry, the one or more food items to recommend for the consumption by the patient based on the predicted patient nutrition state corresponding to one or more food item profiles, wherein a corresponding predicted patient nutrition state of the patient comprises at least one estimated biomarker level expected to be within at least one desired range if a delivery device executes a therapy dosage and the patient consumes the selected food item.

15. The method of claim 15 further comprising determining, by the processing circuitry, the therapy dosage based at least in part on the one or more selected food items.

16. The method of claim 15, wherein the delivery device executes the therapy information to direct therapy delivery to the patient.

17. The method of claim 14, wherein selecting, by the processing circuitry, the one or more food items further comprises identifying, from amongst a set of food items, the one or more selected food items to recommend based on a comparison between the predicted patient nutrition state and a desired patient nutrition state.

18. The method of claim 14 further comprising training, by the processing circuitry, the trained model by, for each baseline food item of a set of predetermined baseline food items, applying a model to a corresponding baseline food item profile having a predetermined set of nutritional attributes for that baseline food item until satisfaction of an accuracy metric.

19. The method of claim 17, wherein training, by the processing circuitry, the model further comprises:
determining an estimated biomarker level based on the patient consuming a particular baseline food item; and
calibrating the model based on comparing the estimated biomarker level to an actual biomarker level of the patient after the patient consumes the particular baseline food item within a time period.

20. The method of claim 14, wherein selecting, by the processing circuitry, the one or more food items to recommend further comprises generating, for a particular food item, a corresponding predicted patient nutrition state based on the trained model and a set of nutritional attributes for the particular food item.
